SEQUENCE LISTING (4 sequences)

SEQ ID NO: 1 - DNA, 1342 bp, Pseudozyma
  CDS (65)..(1246)
cctccaacaaagaagatcgatctcattgaagaatacgctaaatcacctttgtacgatcctcatcatgaagctcacagcatctttcgctgctttggccacagtgctcttatctgctagcacatttgctgctccaaacccatcaagtgcaaaccaaaacacaatcccattaaacaaaaaacgtgatgctcttttggttccaggttcaaatgaagttgattttaccaaagttcgtgctcatttagatcacgtaaaggcaaaatactcggacaatttacaagcttttgcagcaaacacaggtaacacccatccattgcaatcaaaaacattcaagccattctcaaaaagagcaggaacaggatccgttagcttgacagatattcaaaatgaggaattatggtcaggtccagtcaaatttggtggacaaacaatctatgtcgatttcgatacaggaagtgcagatgtcatcattaaccataacgcttacactccaggatcaactgcaaagaacacaggaaagactttttcaaccgcttacggtgatggtacaaccgcttcaggtcccgtttacactgatacgttctccatcggtggtctaagcgcaaactctgctgctttgggttggtcaaaaaaccaattcttgaccggtgaatcaccaaacaatggtattgctggaatgagttatccatctttggcaactttaggttatccaccattctttgacacattgggcaatgccggtgcattggcaagaaatgttttcactttcacactttcaaaaggagcatctactctctatcttggtggcgttgatccaaaagctggttcgccaaagtatgtcaacgttgattcttcccaaggcttctggactattgacagtggttcgattgctggcgtttcaaccggttccattcaagataccggtaccactgttatcgttgcaccaactgataccgctcaaaacatcttctcaaacttgccaggggtaacgcaatttcaacaagacggcgcttattacggtgcttttaactgcaacagtccaccacaagttaccattcaattgggtgggtactctcaagcattgtccagtgctaccacatcattcgggaccacaaatgacgggcaatgcgttttgagtgttgtaggtgaggatatcggattggacactgtcattttgggggacagctggttgcagaatgtgcacgctgtgtttgaccgtgacaacaaccgggtcggattcagcaagcaatgaatgtaaggttaaaattgatggggttaattgattaaattgattgaaaaaaaaaaaaaaaaaaaaaaaaaaaaaaaaaaaaaaaaaaaaaaaaaa

SEQ ID NO: 2 - protein, 394 aa, Pseudozyma
MKLTASFAALATVLLSASTFAAPNPSSANQNTIPLNKKRDALLVPGSNEVDFTKVRAHLDHVKAKYSDNLQAFAANTGNTHPLQSKTFKPFSKRAGTGSVSLTDIQNEELWSGPVKFGGQTIYVDFDTGSADVIINHNAYTPGSTAKNTGKTFSTAYGDGTTASGPVYTDTFSIGGLSANSAALGWSKNQFLTGESPNNGIAGMSYPSLATLGYPPFFDTLGNAGALARNVFTFTLSKGASTLYLGGVDPKAGSPKYVNVDSSQGFWTIDSGSIAGVSTGSIQDTGTTVIVAPTDTAQNIFSNLPGVTQFQQDGAYYGAFNCNSPPQVTIQLGGYSQALSSATTSFGTTNDGQCVLSVVGEDIGLDTVILGDSWLQNVHAVFDRDNNRVGFSKQ

SEQ ID NO: 3 - DNA, 27 bp, artificial sequence
  Primer
acnganatncanaanganganntntgg

SEQ ID NO: 4 - DNA, 19 bp, artificial sequence
  Primer
ggccgcaggaatttttttt

(19) United States
(12) Patent Application Publication
(10) Pub. No.: US 2003/0032165 A1
(43) Pub. Date: Feb. 13, 2003

(54) Aspartic acid proteases and nucleic acids encoding same

(75) Inventors: Wenping Wu, Beijing (CN); Peter Rahbek Ostergaard, Virum (DK); Markus Sakari Kauppinen, Smorum (DK)

Correspondence Address: NOVOZYMES NORTH AMERICA, INC., 500 FIFTH AVENUE, SUITE 1600, NEW YORK, NY, 10110, US

(73) Assignee: Novozymes A/S, Bagsvaerd (DK)

(21) Appl. No.: 09/879,389

(22) Filed: Jun. 12, 2001

Related U.S. Application Data
(60) Provisional application No. 60/211,561, filed on Jun. 15, 2000.

(30) Foreign Application Priority Data
Jun. 13, 2000 (DK) 2000 00904

(51) Int. Cl. C12Q 1/68; C12Q 1/37; C07H 21/04; C12N 9/64; C12P 21/02; C12N 5/06

(52) U.S. Cl. 435/226; 435/69.1; 435/320.1; 435/325; 435/6; 536/23.2; 435/23

(57) ABSTRACT

The present invention relates to novel isolated aspartic acid proteases and isolated nucleic acid sequences encoding such aspartic acid proteases. The present invention also relates to nucleic acid constructs, vectors and host cells comprising the nucleic acid sequences as well as to methods for producing and using the aspartic acid proteases.

### CROSS-REFERENCE TO RELATED APPLICATIONS

This application claims, under 35 U.S.C. 119, priority or the benefit of Danish application no. PA 2000 00904, filed Jun. 13, 2000, and the benefit of U.S. provisional application No. 60/211,561, filed Jun. 15, 2000, the contents of which are fully incorporated herein by reference.

### FIELD OF THE INVENTION

The present invention relates to novel isolated aspartic acid proteases and isolated nucleic acid sequences encoding such aspartic acid proteases. The present invention also relates to nucleic acid constructs, vectors and host cells comprising the nucleic acid sequences as well as to methods for producing and using the aspartic acid proteases.

### BACKGROUND OF THE INVENTION

Aspartic acid proteases differ importantly from the more intensively studied serine proteases in that the nucleophile that attacks the scissile peptide bond is an activated water molecule rather than the nucleophilic side chain of an amino acid residue. Aspartic acid proteases are so named because Asp residues are ligands of the activated water molecule. The best-known member of the family is pepsin. Examples of other members are chymosin (rennin), Cathepsin D and penicillinopepsin (for an overview, see for example Handbook of Proteolytic Enzymes, Edited by A. J. Barrett, N. D. Rawlings and J. F. Woessner, Academic Press, San Diego, 1998, Chapter 271).

Aspartic acid proteases are widely used industrially, such as in the preparation of food, in the leather industry, in the production of protein hydrolysates and in the wine and brewing industry.

Berka et al. disclose a gene encoding the aspartic acid Aspergillopepsin A from Aspergillus awamori (R. M. Berka et al. Gene, 96, 313 (1990)).

Berka et al. also disclose a gene encoding the aspartic acid protease Aspergillopepsin O from Aspergillus oryzae (R. M. Berka et al. Gene, 125, 195-198 (1993)).

The cloning of a gene encoding an aspartic acid protease from Aspergillus oryzae was disclosed by Gomi et al. Biosci. Biotech. Biochem. 57, 1095-1100 (1993).

There are, however, still a need for novel aspartic acid proteases having an increased/broader pH-stability. This requirement is met by the novel aspartic acid proteases disclosed herein.

### SUMMARY OF THE INVENTION

Thus, in a first aspect the present invention relates to an isolated aspartic acid protease, selected from the group consisting of:

(a) an aspartic acid protease having an amino acid sequence which has at least 40% identity with the amino acid sequence shown as amino acids 1 to 300 of SEQ ID NO:2;

(b) an aspartic acid protease which is encoded by a nucleic acid sequence which hybridizes under low stringency conditions with

(i) a complementary strand of the nucleic acid sequence shown as nucleotides 347 to 1246 of SEQ ID NO:1, or

(ii) a subsequence of (i) of at least 100 nucleotides;

(c) an aspartic acid protease encoded by the aspartic acid protease encoding part of the DNA sequence cloned into a plasmid present in Escherichia coli DSM 13470, or a variant thereof having at least 40% identity to said aspartic acid protease;

(d) an aspartic acid protease having a relative activity of at least 0.75 throughout the pH range from 3 to 4, when tested at 37° C. for 30 min in the “BSA-BCA pH-activity assay” described herein;

(e) an aspartic acid protease having a residual activity of at least 0.70 after incubation for 2 hours at 37° C. throughout the pH range from 3 to 7 and subsequently tested at 37° C. for 30 min at pH 3 in the “BSA-BCA pH-stability assay” described herein; and

(f) an aspartic acid protease having a specific activity (units/mg protease) of at least 4.0 when tested for 30 min at pH 3 and 37° C. in the “BSA-BCA assay” described herein.

In a second aspect the present invention relates to an isolated nucleic acid sequence comprising a nucleic acid sequence which encodes for the aspartic acid protease of the invention.

In a third aspect the present invention relates to an isolated nucleic acid sequence encoding an aspartic acid protease, selected from the group consisting of:

(a) a nucleic acid sequence having at least 40% identity with the nucleic acid sequence shown as nucleotides 347 to 1246 of SEQ ID NO:1;

(b) a nucleic acid sequence which hybridizes under low stringency conditions with

(i) a complementary strand of the nucleic acid sequence shown as nucleotides 347 to 1246 of SEQ ID NO:1, or

(ii) a subsequence of (i) of at least 100 nucleotides; and

(c) the aspartic acid encoding part of the DNA sequence which has been cloned into a plasmid present in Escherichia coli DSM 13470, or a variant thereof having at least 40% identity to said DNA sequence;

or an isolated nucleic acid sequence which is the complementary strand of (a), (b) or (c).

In a fourth aspect the present invention relates a nucleic acid construct comprising the nucleic acid sequence of the invention operably linked to one or more control sequences capable of directing the expression of the aspartic acid protease in a suitable expression host.

In a fifth aspect the present invention relates to a recombinant expression vector comprising the nucleic acid construct of the invention, a promoter, and transcriptional and translational stop signals.

In a sixth aspect the present invention relates to a recombinant host cell comprising the nucleic acid construct of the invention.

Further aspects of the present invention relates to methods for producing the aspartic acid protease of the invention, to use of such proteases for facilitating nitrogen uptake during yeast fermentation, in particular in alcohol production, as well as to methods for facilitating nitrogen uptake during yeast fermentation, in particular in alcohol production.

### BRIEF DESCRIPTION OF THE DRAWINGS

FIG. 1 illustrates the pH activity profile of the aspartic acid protease of the invention. Detailed information regarding the experimental conditions is given in Example 2.

FIG. 2 illustrates the pH stability profile of the aspartic acid protease of the invention. Detailed information regarding the experimental conditions is given in Example 3.

FIG. 3 illustrates the temperature profile of the aspartic acid protease of the invention. Detailed information regarding the experimental conditions is given in Example 4.

### DETAILED DESCRIPTION OF THE INVENTION

Use of Aspartic Acid Proteases in the Alcohol Industry

Aspartic acid proteases digest protein in plant materials (such as corn flower, wheat bran, rice sheath, sweet potato flower, sorghum, etc.) used in alcohol fermentation into free amino acids. Such free amino acids function as nutrients for the yeast, thereby enhancing the growth of the yeast and, consequently, the production of ethanol.

Especially in simultaneous saccharification and fermentation (SSF) processes for production of ethanol from starch, or grains (e.g. whole corn, wheat and barley) the aspartic protease according to the invention can secure optimal fermentations performed at high dry solid contents and additionally secure efficient downstream-processes like distillations, centrifuging, evaporation of thin stillage, and drying of DDG by reducing the fouling and scaling tendency of proteins from the broth when operating at high dry solid contents.

In addition to the enhanced growth of the yeast several additional advantages are contemplated:

The fermentation process will cause less pollution as ammonium sulfate may be used as a nitrogen source during fermentation; and

The time needed before fermentation is completed will be reduced and the amount of ethanol produced will be higher (typically 2-4%).

Application of the aspartic protease could be in any of the following three stages: Before saccharification; after saccharification and inoculation; or approximately 6 hours after inoculation.

Aspartic Acid Proteases of the Invention

For the purposes of the present invention the term “aspartic acid protease” is used in its conventional meaning (see, for example, Handbook of Proteolytic Enzymes, Edited by A. J. Barrett, N. D. Rawlings and J. F. Woessner, Academic Press, San Diego, 1998, Chapter 270).

In a first embodiment, the present invention relates to isolated aspartic acid proteases having an amino acid sequence which has a degree of identity to the amino acid sequence of the mature part of SEQ ID NO:2 of at least 40%, such as at least 50%, e.g. at least 60%, preferably at least 65%, more preferably at least 70%, even more preferably at least 75%, most preferably at least 80%, and even most preferably at least 85%. In a particular preferred embodiment of the invention the isolated aspartic acid protease has an amino acid sequence which has a degree of identity to the amino acid sequence of the mature part of SEQ ID NO:2 of at least 90%, such as at least 91%, preferably at least 92%, such as at least 93%, more preferably at least 94%, such as at least 95%, even more preferably at least 96%, such as at least 97%, most preferably at least 98%, and even most preferably at least 99% (hereinafter “homologous aspartic acid proteases”). In a preferred embodiment, the homologous proteases have an amino acid sequence which differs by five amino acids, preferably by four amino acids, more preferably by three amino acids, even more preferably by two amino acids, and most preferably by one amino acid from the amino acid sequence of SEQ ID NO:2.

Alignment of sequences and calculation of identity scores can be obtained by the GAP routine of the Genetics Computer Group (GCG) package, version 10.0, using the following parameters: Gap creation penalty=8, gap extension penalty=2, and all other parameters kept at their default values.

By performing such alignments, the following identities (in percentage) between the protease precursor from Pseudozyma sp. (i.e. the sequence consisting of amino acids −94 to 300 of SEQ ID NO:2) and various known aspartic acid proteases were found:

| | % identity to |
|---|---|
| | SEQ ID NO: 2 |
| P. janthicellum/Penicillopepsin, P00798 | 35.3 |
| A. satoi/Aspergillopepsin A, Q12567 | 35.4 |
| A. oryzae/Aspergillopepsin O, Q00249 | 31.5 |
| A. fumigatus/Aspergillopepsin F, P41748 | 31.6 |
| A. oryzae/Aspergillopepsin A, Q06902 | 31.5 |
| C. gloesporioides/aspartic protease, Q00895 | 31.2 |

Preferably, the aspartic acid proteases of the present invention comprise the amino acid sequence of the mature part of SEQ ID NO:2, or an allelic variant thereof. In a more preferred embodiment, the aspartic acid protease of the present invention comprise the amino acid sequence of the mature part of SEQ ID NO:2. In another preferred embodiment, the aspartic acid protease of the present invention consists of the amino acid sequence of the mature part of SEQ ID NO:2 or a fragment thereof, wherein the fragment has protease activity. A fragment of SEQ ID NO:2 is an aspartic acid protease having one or more amino acids deleted from the amino and/or carboxy terminus of this amino acid sequence. In a most preferred embodiment, the aspartic acid protease of the invention consists of the amino acid sequence of the mature part of SEQ ID NO:2.

An allelic variant denotes any of two or more alternative forms of a gene occupying the same chomosomal locus. Allelic variation arises naturally through mutation, and may result in phenotypic polymorphism within populations. Gene mutations can be silent (no change in the encoded aspartic acid protease) or may encode aspartic acid proteases having altered amino acid sequences. The term allelic variant of an aspartic acid protease is an aspartic acid protease encoded by an allelic variant of a gene.

The amino acid sequences of the homologous aspartic acid proteases may differ from the amino acid sequence of the mature part of SEQ ID NO:2 by an insertion or deletion of one or more amino acid residues and/or the substitution of one or more amino acid residues by different amino acid residues. Preferably, amino acid changes are of aminor nature, that is conservative amino acid substitutions that do not significantly affect the folding and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain. Examples of conservative substitutions are within the group of basic amino acids (such as arginine, lysine and histidine), acidic amino acids (such as glutamic acid and aspartic acid), polar amino acids (such as glutamine and asparagine), hydrophobic amino acids (such as leucine, isoleucine and valine), aromatic amino acids (such as phenylalanine, tryptophan and tyrosine), and small amino acids (such as glycine, alanine, serine, threonine and methionine). Amino acid substitutions which do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

In a second embodiment, the present invention relates to isolated aspartic acid proteases which are encoded by nucleic acid sequences which hybridize under low stringency conditions, more preferably medium stringency conditions, and most preferably high stringency conditions, with an oligonucleotide probe which hybridizes under the same conditions with the nucleic acid sequence of SEQ ID NO:1 or its complementary strand (J. Sambrook, E. F. Fritsch, and T. Maniatus, 1989, Molecular Cloning, A Laboratory Manual, 2d edition, Cold Spring Harbor, N.Y. ).

The nucleic acid sequence of SEQ ID NO:1, or a subsequence thereof, as well as the amino acid sequence of SEQ ID NO:2, or a partial sequence thereof, may be used to design an oligonucleotide probe to identify and clone DNA encoding aspartic acid proteases from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 15, preferably at least 25, and more preferably at least 40 nucleotides in length. Longer probes can also be used. Both DNA and RNA probes can be used. The probes are typically labeled for detecting the corresponding gene (for example, with ³²P, ³H, ³⁵S, biotin, or avidin).

Thus, a genomic, cDNA or combinatorial chemical library prepared from such other organisms may be screened for DNA which hybridizes with the probes described above and which encodes an aspartic acid protease. Genomic or other DNA from such other organisms may be separated by agarose or polyacrylamide gel electrophoresis, or other separation techniques. DNA from the libraries or the separated DNA may be transferred to and immobilized on nitrocellulose or another suitable carrier material. In order to identify a clone or DNA which is homologous with SEQ ID NO:1, the carrier material is used in a Southern blot. Hybridization indicates that the nucleic acid sequence hybridizes to the oligonucleotide probe corresponding to the aspartic acid encoding part of the nucleic acid sequence shown in SEQ ID NO:1, under low to high stringency conditions (i.e., prehybridization and hybridization at 42° C. in 5× SSPE, 0.3% SDS, 200 μg/ml sheared and denatured salmon sperm DNA, and either 25, 35 or 50% formamide for low, medium and high stringencies, respectively), following standard Southern blotting procedures. The carrier material is finally washed three times each for 30 minutes using 2× SSC, 0.2% SDS preferably at least 50° C. (very low stringency), more preferably at least 55° C. (low stringency), more preferably at least 60° C. (medium stringency), more preferably at least 65° C. (medium-high stringency), even more preferably at least 70° C. (high stringency), and most preferably at least 75° C. (very high stringency). Molecules to which the oligonucleotide probe hybridizes under these conditions are detected using X-ray film. In a further interesting embodiment of the invention the aspartic acid protease of the invention has a relative activity of at least 0.75, preferably at least 0.80, such as at least 0.85, in particular at least 0.90, throughout the pH range from 3 to 4, when tested at 37° C. for 30 min in the “BSA-BCA pH-activity assay” described herein.

In a still further embodiment of the invention the aspartic acid protease of the invention has a residual activity of at least 0.70, preferably at least 0.75, after incubation for 2 hours at 37° C. throughout the pH range from 3 to 7 and subsequently tested at 37° C. for 30 min at pH 3 in the “BSA-BCA pH-stability assay” described herein.

In another interesting embodiment of the invention the aspartic acid protease of the invention has a specific activity (units/mg protease) of at least 4.5, such as at least 5.0, preferably at least 5.5, more preferably at least 6.0, in particular at least 6.5, when tested for 30 min at pH 3 and 37° C. in the “BSA-BCA assay” described herein.

Further, it is preferred that the aspartic acid protease of the invention has a relative activity of at least 0.50, preferably at least 0.60, throughout the temperature range from 35 to 55° C., such as throughout the temperature range from 40 to 55° C., when tested at pH 3 for 30 min in the “BSA-BCA temperature-activity assay” described herein.

A description of the above-mentioned assays is given in the experimental part, herein.

Moreover, aspartic acid proteases which are also considered as being within the scope of the present invention, are aspartic acid proteases, preferably in a purified form, having immunochemical identity or partial immunochemical identity to the aspartic acid protease having the amino acid sequence of SEQ ID NO:2. The immunochemical properties are determined by immunological cross-reaction identity tests by the well-known Ouchterlony double immunodiffusion procedure. Specifically, an antiserum containing antibodies which are immunoreactive or bind to epitopes of the aspartic acid protease having the amino acid sequence of SEQ ID NO:2 are prepared by immunizing rabbits (or other rodents) according to the procedure described by Harboe and Ingild, In N. H. Axelsen, J. Krøll, and B. Weeks, editors, A Manual of Quantitative Immunoelectrophoresis, Blackwell Scientific Publications, 1973, Chapter 23, or Johnstone and Thorpe, Immunochemistry in Practice, Blackwell Scientific Publications, 1982 (more specifically pages 27-31). An aspartic acid protease having immunochemical identity is an aspartic acid protease, which reacts with the antiserum in an identical fashion such as total fusion of precipitates, identical precipitate morphology, and/or identical electrophoretic mobility using a specific immunochemical technique. A further explanation of immunochemical identity is described by Axelsen, Bock, and Krøll, In N. H. Axelsen, J. Krøll, and B. Weeks, editors, A Manual of Quantitative Immunoelectrophoresis, Blackwell Scientific Publications, 1973, Chapter 10. An aspartic acid protease having partial immunochemical identity is an aspartic acid protease which reacts with the antiserum in a partially identical fashion such as partial fusion of precipitates, partially identical precipitate morphology, and/or partially identical electrophoretic mobility using a specific immunochemical technique. A further explanation of partial immunochemical identity is described by Bock and Axelsen, In N. H. Axelsen, J. Krøll, and B. Weeks, editors, A Manual of Quantitative Immunoelectrophoresis, Blackwell Scientific Publications, 1973, Chapter 11.

Aspartic acid proteases of the invention may be obtained from microorganisms of any genus. In an interesting embodiment, these aspartic acid proteases may be obtained from a bacterial source. For example, these aspartic acid proteases may be obtained from a gram positive bacterium such as a Bacillus strain, e.g., Bacillus alkalophilus, Bacillus amyloliquefaciens, Bacillus brevis, Bacillus circulans, Bacillus coagulans, Bacillus lautus, Bacillus lentus, Bacillus licheniformis, Bacillus megaterium, Bacillus stearothermophilus, Bacillus subtilis, or Bacillus thuringiensis; or a Streptomyces strain, e.g., Streptomyces lividans or Streptomyces murinus; or from a gram negative bacterium, e.g., E. coli or Pseudomonas sp.

In another interesting embodiment the aspartic acid proteases of the invention may be obtained from a fungal source, and more preferably from a yeast strain such as a Candida, Kluyveromyces, Phaffia, Pichia, Saccharomyces, Schizosaccharomyces, or Yarrowia strain; or a filamentous fungal strain such as an Acremonium, Aspergillus, Aureobasidium, Cryptococcus, Filibasidium, Fusarium, Humicola, Magnaporthe, Mucor, Myceliophthora, Neocallimastix, Neurospora, Paecilomyces, Penicillium, Piromyces, Schizophyllum, Talaromyces, Thermoascus, Thielavia, Tolypocladium, or Trichoderma strain.

In a preferred embodiment, the aspartic acid proteases are obtained from a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis strain.

In another preferred embodiment, the aspartic acid proteases are obtained from an Aspergillus aculeatus, Aspergillus awamori, Aspergillus foetidus, Aspergillus japonicus, Aspergillus nidulans, Aspergillus niger, Aspergillus oryzae, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Trichoderma harzianum, Trichoderma koningii, Trichoderma longibrachiatum, Trichoderma reesei, or Trichoderma viride strain.

In a particular interesting embodiment, the aspartic acid protease is obtained from the genus Pseudozyma, preferably from the species Pseudozyma sp.

We have shown that Pseudozyma rugulosa, Pseudozyma tsukubaensis, Pseudozyma antartica, Pseudozyma aphidis, and Pseudozyma flocculosa all produce aspartic acid protease.

The present inventors have isolated the gene encoding the aspartic acid protease of the invention from Pseudozyma sp. and inserted it into E. coli DH10B. The E. coli strain harboring the gene was deposited according to the Budapest Treaty on the International Recognition of the Deposits of Microorganisms for the Purpose of Patent Procedures on May 2, 2000 at the Deutsche Sammlung von Mikroorganismen und Zellkulturen GmbH, Mascheroder Weg 1 B, D-38124 Braunschweig, Germany, and designated the accession No. DSM 13470.

Therefore, in a further embodiment of the present invention the aspartic acid protease of the invention is a protease encoded by the aspartic acid protease encoding part of the DNA sequence cloned into a plasmid present in Escherichia coli DSM 13470, or a variant thereof having at least 40% identity to said aspartic acid protease. With respect to the variant it is preferred that the variant aspartic acid protease has at least 40%, such as at least 50%, e.g. at least 60%, preferably at least 70%, more preferably at least 75%, even more preferably at least 80%, most preferably at least 85% identity with the aspartic acid protease encoded by the glucanotransferase encoding part of the DNA sequence cloned into a plasmid present in Escherichia coli DSM 13470. In a particular preferred embodiment of the invention the aspartic acid protease variant has at least 90%, such as at least 91%, preferably at least 92%, such as at least 93%, more preferably at least 94%, such as at least 95%, even more preferably at least 96%, such as at least 97%, most preferably at least 98%, and even most preferably at least 99% identity with the aspartic acid protease encoded by the aspartic acid protease encoding part of the DNA sequence cloned into a plasmid present in Escherichia coli DSM 13470.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of the above-mentioned species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such aspartic acid proteases may be identified and obtained from other sources including microorganisms isolated from nature (e.g., plant, soil, composts, water, etc.) using the above-mentioned probes. Techniques for isolating microorganisms from natural habitats are well known in the art. The nucleic acid sequence may then be derived by similarly screening a genomic or cDNA library of another microorganism. Once a nucleic acid sequence encoding an aspartic acid protease has been detected with the probe(s), the sequence may be isolated or cloned by utilizing techniques which are known to those of ordinary skill in the art (see, e.g., Sambrook et al., 1989, supra).

For purposes of the present invention, the term “obtained from” as used herein in connection with a given source shall mean that the aspartic acid protease is produced by the source or by a cell in which a gene from the source has been inserted.

As defined herein, an “isolated” aspartic acid protease is an aspartic acid protease which is essentially free of other polypeptides, e.g., at least about 20% pure, preferably at least about 40% pure, more preferably about 60% pure, even more preferably about 80% pure, most preferably about 90% pure, and even most preferably about 95% pure, as determined by SDS-PAGE.

Nucleic Acid Sequences

The present invention also relates to isolated nucleic acid sequences which encode an aspartic acid protease of the present invention.

In one interesting embodiment, the nucleic acid sequence has an identity with the nucleic acid sequence shown as nucleotides 347 to 1246 of SEQ ID NO: 1 of at least 40%, such as at least 50%, e.g. at least 60%, preferably at least 65%, more preferably at least 70%, even more preferably at least 75%, most preferably at least 80%, and even most preferably at least 85%. In a particular preferred embodiment of the invention the nucleic acid sequence has a degree of identity to the nucleic acid sequence shown as nucleotides 347 to 1246 of SEQ ID NO: 1 of at least 90%, such as at least 91%, preferably at least 92%, such as at least 93%, more preferably at least 94%, such as at least 95%, even more preferably at least 96%, such as at least 97%, most preferably at least 98%, and even most preferably at least 99%. In another interesting embodiment of the invention the nucleic acid sequence comprises the nucleic acid sequence shown as nucleotides 347 to 1246 of SEQ ID NO:1, an allelic variant thereof, or a fragment thereof capable of encoding an aspartic acid protease according to the invention. Obviously, the nucleic acid sequence may consist of the nucleic acid sequence shown as nucleotides 347 to 1246 of SEQ ID NO:1.

In another preferred embodiment, the nucleic acid sequence is the aspartic acid encoding part of the DNA sequence which has been cloned into a plasmid present in Escherichia coli DSM 13470 or a variant thereof having at least 40% identity to said DNA sequence. With respect to the variant it is preferred that the variant DNA sequence has at least 50%, such as at least 60%, e.g. at least 65%, preferably at least 70%, more preferably at least 75%, even more preferably at least 80%, most preferably at least 85% identity with the aspartic acid protease encoding part of the DNA sequence which has been cloned into a plasmid present in Escherichia coli DSM 13470. In a particular preferred embodiment of the invention the variant DNA sequence has at least 90%, such as at least 91%, preferably at least 92%, such as at least 93%, more preferably at least 94%, such as at least 95%, even more preferably at least 96%, such as at least 97%, most preferably at least 98%, and even most preferably at least 99% identity with the aspartic acid protease encoding part of the DNA sequence which has been cloned into a plasmid present in Escherichia coli DSM 13470.

The present invention also encompasses nucleic acid sequences which encode an aspartic acid protease having the amino acid sequence of SEQ ID NO:2, which differ from SEQ ID NO: 1 by virtue of the degeneracy of the genetic code.

In a preferred embodiment, the nucleic acid sequence encodes an aspartic acid protease obtained from Pseudozyma and in a more preferred embodiment, the nucleic acid sequence is obtained from Pseudozyma sp.

In another more preferred embodiment, the nucleic acid sequence is the sequence contained in plasmid pYES 2.0, which is contained in Escherichia coli DH10B. The present invention also encompasses nucleic acid sequences which encode an aspartic acid protease having the amino acid sequence of SEQ ID NO:2, which differ from SEQ ID NO:1 by virtue of the degeneracy of the genetic code.

The techniques used to isolate or clone a nucleic acid sequence encoding an aspartic acid protease are known in the art and include isolation from genomic DNA, preparation from cDNA, or a combination thereof. The cloning of the nucleic acid sequences of the present invention from such genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligated activated transcription (LAT) and nucleic acid sequence-based amplification (NASBA) may be used. The nucleic acid sequence may be cloned from a strain of Pseudozyma, or another or related organism and thus, for example, may be an allelic or species variant of the aspartic acid protease encoding region of the nucleic acid sequence.

The term “isolated nucleic acid sequence” as used herein refers to a nucleic acid sequence which is essentially free of other nucleic acid sequences, e.g., at least about 20% pure, preferably at least about 40% pure, more preferably at least about 60% pure, even more preferably at least about 80% pure, and most preferably at least about 90% pure as determined by agarose electrophoresis. For example, an isolated nucleic acid sequence can be obtained by standard cloning procedures used in genetic engineering to relocate the nucleic acid sequence from its natural location to a different site where it will be reproduced. The cloning procedures may involve excision and isolation of a desired nucleic acid fragment comprising the nucleic acid sequence encoding the aspartic acid protease, insertion of the fragment into a vector molecule, and incorporation of the recombinant vector into a host cell where multiple copies or clones of the nucleic acid sequence will be replicated. The nucleic acid sequence may be of genomic, cDNA, RNA, semisynthetic, synthetic origin, or any combinations thereof.

Modification of a nucleic acid sequence encoding an aspartic acid protease of the present invention may be necessary for the synthesis of aspartic acid protease substantially similar to the aspartic acid protease. The term “substantially similar” to the aspartic acid protease refers to non-naturally occurring forms of the aspartic acid protease. These aspartic acid proteases may differ in some engineered way from the aspartic acid protease isolated from its native source. For example, it may be of interest to synthesize variants of the aspartic acid protease where the variants differ in specific activity, thermostability, pH optimum, or the like using, e.g., site-directed mutagenesis. The analogous sequence may be constructed on the basis of the nucleic acid sequence presented as the aspartic acid protease encoding part of SEQ ID NO:1, e.g., a subsequence thereof, and/or by introduction of nucleotide substitutions which do not give rise to another amino acid sequence of the aspartic acid protease encoded by the nucleic acid sequence, but which corresponds to the codon usage of the host organism intended for production of the enzyme, or by introduction of nucleotide substitutions which may give rise to a different amino acid sequence. For a general description of nucleotide substitution, see, e.g., Ford et al., 1991, Protein Expression and Purification 2: 95-107.

It will be apparent to those skilled in the art that such substitutions can be made outside the regions critical to the function of the molecule and still result in an active aspartic acid protease. Amino acid residues essential to the activity of the aspartic acid protease encoded by the isolated nucleic acid sequence of the invention, and therefore preferably not subject to substitution, may be identified according to procedures known in the art, such as site-directed mutagenesis or alanine-scanning mutagenesis (see, e.g., Cunningham and Wells, 1989, Science 244: 1081-1085). In the latter technique, mutations are introduced at every positively charged residue in the molecule, and the resultant mutant molecules are tested for enzyme activity to identify amino acid residues that are critical to the activity of the molecule. Sites of substrate-enzyme interaction can also be determined by analysis of the three-dimensional structure as determined by such techniques as nuclear magnetic resonance analysis, crystallography or photoaffinity labelling (see, e.g., de Vos et al., 1992, Science 255: 306-312; Smith et al., 1992, Journal of Molecular Biology 224: 899-904; Wlodaver et al., 1992, FEBS Letters 309: 59-64).

The present invention also relates to isolated nucleic acid sequences encoding an aspartic acid protease of the present invention, which hybridize under low stringency conditions, more preferably medium stringency conditions, and most preferably high stringency conditions, with an oligonucleotide probe which hybridizes under the same conditions with the nucleic acid sequence of SEQ ID NO: 1 or its complementary strand; or allelic variants and subsequences thereof (Sambrook et al., 1989, supra).

Nucleic Acid Constructs

The present invention also relates to nucleic acid constructs comprising a nucleic acid sequence of the present invention operably linked to one or more control sequences, which direct the expression of the coding sequence in a suitable host cell under conditions compatible with the control sequences. Expression will be understood to include any step involved in the production of the aspartic acid protease including, but not limited to, transcription, post-transcriptional modification, translation, post-translational modification, and secretion.

“Nucleic acid construct” is defined herein as a nucleic acid molecule, either single- or double-stranded, which is isolated from a naturally occurring gene or which has been modified to contain segments of nucleic acid which are combined and juxtaposed in a manner which would not otherwise exist in nature. The term nucleic acid construct is synonymous with the term expression cassette when the nucleic acid construct contains all the control sequences required for expression of a coding sequence of the present invention. The term “coding sequence” as defined herein is a sequence, which is transcribed into mRNA and translated into an aspartic acid protease of the present invention. The boundaries of the coding sequence are generally determined by a ribosome binding site (prokaryotes) or by the ATG start codon (eukaryotes) located just upstream of the open reading frame at the 5′ end of the mRNA and a transcription terminator sequence located just downstream of the open reading frame at the 3′ end of the mRNA. A coding sequence can include, but is not limited to, DNA, cDNA, and recombinant nucleic acid sequences.

An isolated nucleic acid sequence encoding an aspartic acid protease of the present invention may be manipulated in a variety of ways to provide for expression of the aspartic acid protease. Manipulation of the nucleic acid sequence encoding an aspartic acid protease prior to its insertion into a vector may be desirable or necessary depending on the expression vector. The techniques for modifying nucleic acid sequences utilizing cloning methods are well known in the art.

The term “control sequences” is defined herein to include all components, which are necessary or advantageous for the expression of an aspartic acid protease of the present invention. Each control sequence may be native or foreign to the nucleic acid sequence encoding the aspartic acid protease. Such control sequences include, but are not limited to, a leader, a polyadenylation sequence, a propeptide sequence, a promoter, a signal sequence, and a transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding an aspartic acid protease. The term “operably linked” is defined herein as a configuration in which a control sequence is appropriately placed at a position relative to the coding sequence of the DNA sequence such that the control sequence directs the production of an aspartic acid protease.

The control sequence may be an appropriate promoter sequence, a nucleic acid sequence which is recognized by a host cell for expression of the nucleic acid sequence. The promoter sequence contains transcriptional control sequences, which mediate the expression of the aspartic acid protease. The promoter may be any nucleic acid sequence which shows transcriptional activity in the host cell of choice including mutant, truncated, and hybrid promoters, and may be obtained from genes encoding extracellular or intracellular aspartic acid proteases either homologous or heterologous to the host cell.

Examples of suitable promoters for directing the transcription of the nucleic acid constructs of the present invention, especially in a bacterial host cell, are the promoters obtained from the E. coli lac operon, the Streptomyces coelicolor agarase gene (dagA), the Bacillus subtilis levansucrase gene (sacB), the Bacillus licheniformis alpha-amylase gene (amyL), the Bacillus stearothermophilus maltogenic amylase gene (amyM), the Bacillus amyloliquefaciens alpha-amylase gene (amyQ), the Bacillus licheniformis penicillinase gene (penP), the Bacillus subtilis xylA and xylB genes, and the prokaryotic beta-lactamase gene (Villa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs of the present invention in a filamentous fungal host cell are promoters obtained from the genes encoding Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium oxysporum trypsin-like protease (U.S. Pat. No. 4,288,627), and mutant, truncated, and hybrid promoters thereof. Particularly preferred promoters for use in filamentous fungal host cells are the TAKA amylase, NA2-tpi (a hybrid of the promoters from the genes encoding Aspergillus niger neutral alpha-amylase and Aspergillus oryzae triose phosphate isomerase), and glaA promoters.

In a yeast host, useful promoters are obtained from the Saccharomyces cerevisiae enolase (ENO-1) gene, the Saccharomyces cerevisiae galactokinase gene (GAL1), the Saccharomyces cerevisiae alcohol dehydrogenase/glyceraldehyde-3-phosphate dehydrogenase genes (ADH2/GAP), and the Saccharomyces cerevisiae 3-phosphoglycerate kinase gene. Other useful promoters for yeast host cells are described by Romanos et al., 1992, Yeast 8: 423-488.

In a mammalian host cell, useful promoters include viral promoters such as those from Simian Virus 40 (SV40), Rous sarcoma virus (RSV), adenovirus, bovine papilloma virus (BPV), and human cytomegalovirus (CMV).

The control sequence may also be a suitable transcription terminator sequence, a sequence recognized by a host cell to terminate transcription. The terminator sequence is operably linked to the 3′ terminus of the nucleic acid sequence encoding the asprtic acid protease. Any terminator, which is functional in the host cell of choice may be used in the present invention.

Preferred terminators for filamentous fungal host cells are obtained from the genes encoding Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes encoding Saccharomyces cerevisiae enolase, Saccharomyces cerevisiae cytochrome C (CYC1), or Saccharomyces cerevisiae glyceraldehyde-3-phosphate dehydrogenase. Other useful terminators for yeast host cells are described by Romanos et al., 1992, supra. Terminator sequences are well known in the art for mammalian host cells.

The control sequence may also be a suitable leader sequence, a nontranslated region of an mRNA which is important for translation by the host cell. The leader sequence is operably linked to the 5′ terminus of the nucleic acid sequence encoding the aspartic acid protease. Any leader sequence, which is functional in the host cell of choice may be used in the present invention.

Preferred leaders for filamentous fungal host cells are obtained from the genes encoding Aspergillus oryzae TAKA amylase and Aspergillus nidulans triose phosphate isomerase.

Suitable leaders for yeast host cells are obtained from the Saccharomyces cerevisiae enolase (ENO-1) gene, the Saccharomyces cerevisiae 3-phosphoglycerate kinase gene, the Saccharomyces cerevisiae alpha-factor, and the Saccharomyces cerevisiae alcohol dehydrogenase/glyceraldehyde-3-phosphate dehydrogenase genes (ADH2/GAP).

The control sequence may also be a polyadenylation sequence, a sequence which is operably linked to the 3′ terminus of the nucleic acid sequence and which, when transcribed, is recognized by the host cell as a signal to add polyadenosine residues to transcribed mRNA.

Any polyadenylation sequence which is functional in the host cell of choice may be used in the present invention.

Preferred polyadenylation sequences for filamentous fungal host cells are obtained from the genes encoding Aspergillus oryzae TAKA amylase, Aspergillus niger glucoamylase, Aspergillus nidulans anthranilate synthase, and Aspergillus niger alpha-glucosidase.

Useful polyadenylation sequences for yeast host cells are described by Guo and Sherman, 1995, Molecular Cellular Biology 15: 5983-5990. Polyadenylation sequences are well known in the art for mammalian host cells.

The control sequence may also be a signal peptide coding region, which codes for an amino acid sequence linked to the amino terminus of the aspartic acid protease which can direct the encoded aspartic acid protease into the cell's secretory pathway. The 5′ end of the coding sequence of the nucleic acid sequence may inherently contain a signal peptide coding region naturally linked in translation reading frame with the segment of the coding region which encodes the secreted aspartic acid protease. Alternatively, the 5′ end of the coding sequence may contain a signal peptide coding region which is foreign to the coding sequence. The foreign signal peptide coding region may be required where the coding sequence does not normally contain a signal peptide coding region. Alternatively, the foreign signal peptide coding region may simply replace the natural signal peptide coding region in order to obtain enhanced secretion of the aspartic acid protease. The signal peptide coding region may be obtained from a glucoamylase or an amylase gene from an Aspergillus species, a lipase or proteinase gene from a Rhizomucor species, the gene for the alpha-factor from Saccharomyces cerevisiae, an amylase or a protease gene from a Bacillus species, or the calf preprochymosin gene. However, any signal peptide coding region which directs the expressed aspartic acid protease into the secretory pathway of a host cell of choice may be used in the present invention.

An effective signal peptide coding region for bacterial host cells is the signal peptide coding region obtained from the maltogenic amylase gene from Bacillus NCIB 11837, the Bacillus stearothermophilus alpha-amylase gene, the Bacillus licheniformis subtilisin gene, the Bacillus licheniformis beta-lactamase gene, the Bacillus stearothermophilus neutral proteases genes (nprT, nprS, nprM), or the Bacillus subtilis prsA gene. Further signal peptides are described by Simonen and Palva, 1993, Microbiological Reviews 57: 109-137.

An effective signal peptide coding region for filamentous fungal host cells is the signal peptide coding region obtained from the Aspergillus oryzae TAKA amylase gene, Aspergillus niger neutral amylase gene, Rhizomucor miehei aspartic proteinase gene, Humicola lanuginosa cellulase gene, or Humicola lanuginosa lipase gene.

Useful signal peptides for yeast host cells are obtained from the genes for Saccharomyces cerevisiae alpha-factor and Saccharomyces cerevisiae invertase. Other useful signal peptide coding regions are described by Romanos et al., 1992, supra.

The control sequence may also be a propeptide coding region, which codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding region may be obtained from the Bacillus subtilis alkaline protease gene (aprE), the Bacillus subtilis neutral protease gene (nprT), the Saccharomyces cerevisiae alpha-factor gene, the Rhizomucor miehei aspartic proteinase gene, or the Myceliophthora thermophila laccase gene (WO 95/33836).

Where both signal peptide and propeptide regions are present at the amino terminus of an aspartic acid protease, the propeptide region is positioned next to the amino terminus of an apartic acid protease and the signal peptide region is positioned next to the amino terminus of the propeptide region.

The nucleic acid constructs of the present invention may also comprise one or more nucleic acid sequences which encode one or more factors that are advantageous for directing the expression of the aspartic acid protease, e.g., a transcriptional activator (e.g., a trans-acting factor), a chaperone, and a processing protease. Any factor that is functional in the host cell of choice may be used in the present invention. The nucleic acids encoding one or more of these factors are not necessarily in tandem with the nucleic acid sequence encoding the aspartic acid protease.

A transcriptional activator is a protein, which activates transcription of a nucleic acid sequence encoding a polypeptide (Kudla et al., 1990, EMBO Journal 9: 1355-1364; Jarai and Buxton, 1994, Current Genetics 26: 2238-244; Verdier, 1990, Yeast 6: 271-297). The nucleic acid sequence encoding an activator may be obtained from the genes encoding Bacillus stearothermophilus NprA (nprA), Saccharomyces cerevisiae heme activator protein 1 (hap1), Saccharomyces cerevisiae galactose metabolizing protein 4 (gal4), Aspergillus nidulans ammonia regulation protein (i), and Aspergillus oryzae alpha-amylase activator (amyR). For further examples, see Verdier, 1990, supra and MacKenzie et al., 1993, Journal of General Microbiology 139: 2295-2307.

A chaperone is a protein which assists another polypeptide to fold properly (Hartl et al., 1994, TIBS 19: 20-25; Bergeron et al., 1994, TIBS 19: 124-128; Demolder et al., 1994, Journal of Biotechnology 32: 179-189; Craig, 1993, Science 260: 1902-1903; Gething and Sambrook, 1992, Nature 355: 33-45; Puig and Gilbert, 1994, Journal of Biological Chemistry 269: 7764-7771; Wang and Tsou, 1993, The FASEB Journal 7: 1515-11157; Robinson et al., 1994, Bio/Technology 1: 381-384; Jacobs et al., 1993, Molecular Microbiology 8: 957-966). The nucleic acid sequence encoding a chaperone may be obtained from the genes encoding Bacillus subtilis GroE proteins, Bacillus subtilis PrsA, Aspergillus oryzae protein disulphide isomerase, Saccharomyces cerevisiae calnexin, Saccharomyces cerevisiae BiP/GRP78, and Saccharomyces cerevisiae Hsp70. For further examples, see Gething and Sambrook, 1992, supra, and Hartl et al., 1994, supra.

A processing protease is a protease that cleaves a propeptide to generate a mature biochemically active polypeptide (Enderlin and Ogrydziak, 1994, Yeast 10: 67-79; Fuller et al., 1989, Proceedings of the National Academy of Sciences USA 86: 1434-1438; Julius et al., 1984, Cell 37: 1075-1089; Julius et al., 1983, Cell 32: 839-852; U.S. Pat. No. 5,702,934). The nucleic acid sequence encoding a processing protease may be obtained from the genes encoding Saccharomyces cerevisiae dipeptidylaminopeptidase, Saccharomyces cerevisiae Kex2, Yarrowia lipolytica dibasic processing endoprotease (xpr6), and Fusarium oxysporum metalloprotease (p45 gene).

It may also be desirable to add regulatory sequences which allow the regulation of the expression of the aspartic acid protease relative to the growth of the host cell. Examples of regulatory systems are those which cause the expression of the gene to be turned on or off in response to a chemical or physical stimulus, including the presence of a regulatory compound. Regulatory systems in prokaryotic systems would include the lac, tac, and trp operator systems. In yeast, the ADH2 system or GAL1 system may be used. In filamentous fungi, the TAKA alpha-amylase promoter, Aspergillus niger glucoamylase promoter, and the Aspergillus oryzae glucoamylase promoter may be used as regulatory sequences. Other examples of regulatory sequences are those, which allow for gene amplification. In eukaryotic systems, these include the dihydrofolate reductase gene, which is amplified in the presence of methotrexate, and the metallothionein genes, which are amplified with heavy metals. In these cases, the nucleic acid sequence encoding the aspartic acid protease would be operably linked with the regulatory sequence.

The present invention also relates to nucleic acid constructs for altering the expression of an endogenous gene encoding an aspartic acid protease of the present invention. The constructs may contain the minimal number of components necessary for altering expression of the endogenous gene. In one embodiment, the nucleic acid constructs preferably contain (a) a targeting sequence, (b) a regulatory sequence, (c) an exon, and (d) a splice-donor site. Upon introduction of the nucleic acid construct into a cell, the construct inserts by homologous recombination into the cellular genome at the endogenous gene site. The targeting sequence directs the integration of elements (a)-(d) into the endogenous gene such that elements (b)-(d) are operably linked to the endogenous gene. In another embodiment, the nucleic acid constructs contain (a) a targeting sequence, (b) a regulatory sequence, (c) an exon, (d) a splice-donor site, (e) an intron, and (f) a splice-acceptor site, wherein the targeting sequence directs the integration of elements (a)-(f) such that elements (b)-(f) are operably linked to the endogenous gene. However, the constructs may contain additional components such as a selectable marker.

In both embodiments, the introduction of these components results in production of a new transcription unit in which expression of the endogenous gene is altered. In essence, the new transcription unit is a fusion product of the sequences introduced by the targeting constructs and the endogenous gene. In one embodiment in which the endogenous gene is altered, the gene is activated. In this embodiment, homologous recombination is used to replace, disrupt, or disable the regulatory region normally associated with the endogenous gene of a parent cell through the insertion of a regulatory sequence, which causes the gene to be expressed at higher levels than evident in the corresponding parent cell. The activated gene can be further amplified by the inclusion of an amplifiable selectable marker gene in the construct using methods well known in the art (see, for example, U.S. Pat. No. 5,641,670). In another embodiment in which the endogenous gene is altered, expression of the gene is reduced.

The targeting sequence can be within the endogenous gene, immediately adjacent to the gene, within an upstream gene, or upstream of and at a distance from the endogenous gene. One or more targeting sequences can be used. For example, a circular plasmid or DNA fragment preferably employs a single targeting sequence, while a linear plasmid or DNA fragment preferably employs two targeting sequences.

The regulatory sequence of the construct can be comprised of one or more promoters, enhancers, scaffold-attachment regions or matrix attachment sites, negative regulatory elements, transcription binding sites, or combinations of these sequences.

The constructs further contain one or more exons of the endogenous gene. An exon is defined as a DNA sequence which is copied into RNA and is present in a mature mRNA molecule such that the exon sequence is in-frame with the coding region of the endogenous gene. The exons can, optionally, contain DNA which encodes one or more amino acids and/or partially encodes an amino acid. Alternatively, the exon contains DNA which corresponds to a 5′ non-encoding region. Where the exogenous exon or exons encode one or more amino acids and/or a portion of an amino acid, the nucleic acid construct is designed such that, upon transcription and splicing, the reading frame is in-frame with the coding region of the endogenous gene so that the appropriate reading frame of the portion of the mRNA derived from the second exon is unchanged.

The splice-donor site of the constructs directs the splicing of one exon to another exon. Typically, the first exon lies 5′ of the second exon, and the splice-donor site overlapping and flanking the first exon on its 3′ side recognizes a splice-acceptor site flanking the second exon on the 5′ side of the second exon. A splice-acceptor site, like a splice-donor site, is a sequence which directs the splicing of one exon to another exon. Acting in conjunction with a splice-donor site, the splicing apparatus uses a splice-acceptor site to effect the removal of an intron.

Expression Vectors

The present invention also relates to recombinant expression vectors comprising a nucleic acid sequence of the present invention, a promoter, and transcriptional and translational stop signals. The various nucleic acid and control sequences described above may be joined together to produce a recombinant expression vector which may include one or more convenient restriction sites to allow for insertion or substitution of the nucleic acid sequence encoding the aspartic acid protease at such sites. Alternatively, the nucleic acid sequence of the present invention may be expressed by inserting the nucleic acid sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), which can be conveniently subjected to recombinant DNA procedures and can bring about the expression of the nucleic acid sequence. The choice of the vector will typically depend on the compatibility of the vector with the host cell into which the vector is to be introduced. The vectors may be linear or closed circular plasmids. The vector may be an autonomously replicating vector, i.e., a vector, which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. The vector system may be a single vector or plasmid or two or more vectors or plasmids which together contain the total DNA to be introduced into the genome of the host cell, or a transposon.

The vectors of the present invention preferably contain one or more selectable markers, which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers, which confer antibiotic resistance such as ampicillin, kanamycin, chloramphenicol or tetracycline resistance. Suitable markers for mammalian cells are the dihydrofolate reductase (dfhr), hygromycin phosphotransferase (hygB), aminoglycoside phosphotransferase II, and phleomycin resistance genes. Suitable markers for yeast host cells are ADE2, HIS3, LEU2, LYS2, MET3, TRP1, and URA3. A selectable marker for use in a filamentous fungal host cell may be selected from the group including, but not limited to, amdS (acetamidase), argB (ornithine carbamoyltransferase), bar (phosphinothricin acetyltransferase), hygB (hygromycin phosphotransferase), niaD (nitrate reductase), pyrG (orotidine-5′-phosphate decarboxylase), sC (sulfate adenyltransferase), trpC (anthranilate synthase), as well as equivalents from other species. Preferred for use in an Aspergillus cell are the amdS and pyrG genes of Aspergillus nidulans or Aspergillus oryzae and the bar gene of Streptomyces hygroscopicus. Furthermore, selection may be accomplished by co-transformation, e.g., as described in WO 91/17243, where the selectable marker is on a separate vector.

The vectors of the present invention preferably contain an element(s) that permits stable integration of the vector into the host cell genome or autonomous replication of the vector in the cell independent of the genome of the cell.

For integration into the host cell genome, the vector may rely on the nucleic acid sequence encoding the aspartic acid protese or any other element of the vector for stable integration of the vector into the genome by homologous or nonhomologous recombination. Alternatively, the vector may contain additional nucleic acid sequences for directing integration by homologous recombination into the genome of the host cell. The additional nucleic acid sequences enable the vector to be integrated into the host cell genome at a precise location(s) in the chromosome(s). To increase the likelihood of integration at a precise location, the integrational elements should preferably contain a sufficient number of nucleic acids, such as 100 to 1,500 base pairs, preferably 400 to 1,500 base pairs, and most preferably 800 to 1,500 base pairs, which are highly homologous with the corresponding target sequence to enhance the probability of homologous recombination. The integrational elements may be any sequence that is homologous with the target sequence in the genome of the host cell. Furthermore, the integrational elements may be non-encoding or encoding nucleic acid sequences. On the other hand, the vector may be integrated into the genome of the host cell by non-homologous recombination.

For autonomous replication, the vector may further comprise an origin of replication enabling the vector to replicate autonomously in the host cell in question. Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus. Examples of origins of replication for use in a yeast host cell are the 2 micron origin of replication, ARS1, ARS4, the combination of ARS1 and CEN3, and the combination of ARS4 and CEN6. The origin of replication may be one having a mutation which makes its functioning temperature-sensitive in the host cell (see, e.g., Ehrlich, 1978, Proceedings of the National Academy of Sciences USA 75: 1433).

More than one copy of a nucleic acid sequence of the present invention may be inserted into the host cell to increase production of the gene product. An increase in the copy number of the nucleic acid sequence can be obtained by integrating at least one additional copy of the sequence into the host cell genome or by including an amplifiable selectable marker gene with the nucleic acid sequence where cells containing amplified copies of the selectable marker gene, and thereby additional copies of the nucleic acid sequence, can be selected for by culturing the cells in the presence of the appropriate selectable agent.

The procedures used to ligate the elements described above to construct the recombinant expression vectors of the present invention are well known to one skilled in the art (see, e.g., Sambrook et al., 1989, supra).

Host Cells

The present invention also relates to recombinant host cells, comprising a nucleic acid sequence of the invention, which are advantageously used in the recombinant production of the aspartic acid proteases. The term “host cell” encompasses any progeny of a parent cell, which is not identical to the parent cell due to mutations that occur during replication.

A vector comprising a nucleic acid sequence of the present invention is introduced into a host cell so that the vector is maintained as a chromosomal integrant or as a self-replicating extra-chromosomal vector. Integration is generally considered to be an advantage as the nucleic acid sequence is more likely to be stably maintained in the cell. Integration of the vector into the host chromosome may occur by homologous or non-homologous recombination as described above.

The choice of a host cell will to a large extent depend upon the gene encoding the aspartic acid protease and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, e.g., Bacillus alkalophilus, Bacillus amyloliquefaciens, Bacillus brevis, Bacillus circulars, Bacillus clausii, Bacillus coagulans, Bacillus lautus, Bacillus lentus, Bacillus licheniformis, Bacillus megaterium, Bacillus stearothermophilus, Bacillus subtilis, and Bacillus thuringiensis; or a Streptomyces cell, e.g., Streptomyces lividans or Streptomyces murinus, or gram negative bacteria such as E. coli and Pseudomonas sp. In a preferred embodiment, the bacterial host cell is a Bacillus lentus, Bacillus licheniformis, Bacillus stearothermophilus or Bacillus subtilis cell. In another preferred embodiment, the Bacillus cell is an alkalophilic Bacillus or an industrial Bacillus.

The introduction of a vector into a bacterial host cell may, for instance, be effected by protoplast transformation (see, e.g., Chang and Cohen, 1979, Molecular General Genetics 168: 111-115), by using competent cells (see, e.g., Young and Spizizin, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thorne, 1987, Journal of Bacteriology 169: 5771-5278).

The host cell may be a eukaryote, such as a mammalian cell, an insect cell, a plant cell or a fungal cell. Useful mammalian cells include Chinese hamster ovary (CHO) cells, HeLa cells, baby hamster kidney (BHK) cells, COS cells, or any number of other immortalized cell lines available, e.g., from the American Type Culture Collection.

In a preferred embodiment, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra). Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g., Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

In a more preferred embodiment, the fungal host cell is a yeast cell. “Yeast” as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g., genera Kluyveromyces, Pichia, and Saccharomyces). The basidiosporogenous yeasts include the genera Leucosporidim, Rhodosporidium, Sporidiobolus, Filobasidium, and Filobasidiella. Yeast belonging to the Fungi Imperfecti are divided into two families, Sporobolomycetaceae (e.g., genera Sporobolomyces and Bullera) and Cryptococcaceae (e.g., genus Candida). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980. The biology of yeast and manipulation of yeast genetics are well known in the art (see, e.g., Biochemistry and Genetics of Yeast, Bacil, M., Horecker, B. J., and Stopani, A. O. M., editors, 2nd edition, 1987; The Yeasts, Rose, A. H., and Harrison, J. S., editors, 2nd edition, 1987; and The Molecular Biology of the Yeast Saccharomyces, Strathem et al., editors, 1981).

In an even more preferred embodiment, the yeast host cell is a cell of a species of Candida, Hansenula, Kluyveromyces, Pichia, Saccharomyces, Schizosaccharomyces, or Yarrowia.

In a most preferred embodiment, the yeast host cell is a Saccharomyces carlsbergensis, Saccharomyces cerevisiae, Saccharomyces diastaticus, Saccharomyces douglasii, Saccharomyces kluyveri, Saccharomyces norbensis or Saccharomyces oviformis cell. In another most preferred embodiment, the yeast host cell is a Kluyveromyces lactis cell. In another most preferred embodiment, the yeast host cell is a Yarrowia lipolytica cell.

In another more preferred embodiment, the fungal host cell is a filamentous fungal cell. “Filamentous fungi” include all filamentous forms of the subdivision Eumycota and Oomycota (as defined by Hawksworth et al., 1995, supra). The filamentous fungi are characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative. In a more preferred embodiment, the filamentous fungal host cell is a cell of a species of, but not limited to, Acremonium, Aspergillus, Fusarium, Humicola, Mucor, Myceliophthora, Neurospora, Penicillium, Thielavia, Tolypocladium, and Trichoderma. In an even more preferred embodiment, the filamentous fungal host cell is an Aspergillus cell.

In another even more preferred embodiment, the filamentous fungal host cell is an Acremonium cell. In another even more preferred embodiment, the filamentous fungal host cell is a Fusarium cell. In another even more preferred embodiment, the filamentous fungal host cell is a Humicola cell. In another even more preferred embodiment, the filamentous fungal host cell is a Mucor cell. In another even more preferred embodiment, the filamentous fungal host cell is a Myceliophthora cell. In another even more preferred embodiment, the filamentous fungal host cell is a Neurospora cell. In another even more preferred embodiment, the filamentous fungal host cell is a Penicillium cell. In another even more preferred embodiment, the filamentous fungal host cell is a Thielavia cell. In another even more preferred embodiment, the filamentous fungal host cell is a Tolypocladium cell. In another even more preferred embodiment, the filamentous fungal host cell is a Trichoderma cell.

In a most preferred embodiment, the filamentous fungal host cell is an Aspergillus awamori, Aspergillus foetidus, Aspergillus japonicus, Aspergillus nidulans, Aspergillus niger or Aspergillus oryzae cell. In another most preferred embodiment, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In an even most preferred embodiment, the filamentous fungal parent cell is a Fusarium venenatum (Nirenberg sp. nov.). In another most preferred embodiment, the filamentous fungal host cell is a Humicola insolens or Humicola lanuginosa cell. In another most preferred embodiment, the filamentous fungal host cell is a Mucor miehei cell. In another most preferred embodiment, the filamentous fungal host cell is a Myceliophthora thermophilum cell. In another most preferred embodiment, the filamentous fungal host cell is a Neurospora crassa cell. In another most preferred embodiment, the filamentous fungal host cell is a Penicillium purpurogenum cell. In another most preferred embodiment, the filamentous fungal host cell is a Thielavia terrestris cell. In another most preferred embodiment, the Trichoderma cell is a Trichoderma harzianum, Trichoderma koningii, Trichoderma longibrachiatum, Trichoderma reesei or Trichoderma viride cell.

Fungal cells may be transformed by a process involving protoplast formation, transformation of the protoplasts, and regeneration of the cell wall in a manner known per se. Suitable procedures for transformation of Aspergillus host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156 and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920. Mammalian cells may be transformed by direct uptake using the calcium phosphate precipitation method of Graham and Van der Eb (1978, Virology 52: 546).

Plants

The present invention also relates to a transgenic plant, plant part, or plant cell which has been transformed with a nucleic acid sequence encoding an aspartic acid protease of the present invention so as to express and produce the aspartic acid protese in recoverable quantities. The aspartic acid protease may be recovered from the plant or plant part.

Alternatively, the plant or plant part containing the recombinant aspartic acid protease may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as festuca, lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers. Also specific plant tissues, such as chloroplast, apoplast, mitochondria, vacuole, peroxisomes, and cytoplasm are considered to be a plant part. Furthermore, any plant cell, whatever the tissue origin, is considered to be a plant part.

Also included within the scope of the present invention are the progeny of such plants, plant parts and plant cells.

The transgenic plant or plant cell expressing an aspartic acid protease of the present invention may be constructed in accordance with methods known in the art. Briefly, the plant or plant cell is constructed by incorporating one or more expression constructs encoding an aspartic acid protease of the present invention into the plant host genome and propagating the resulting modified plant or plant cell into a transgenic plant or plant cell.

Conveniently, the expression construct is a nucleic acid construct which comprises a nucleic acid sequence encoding an aspartic acid protease of the present invention operably linked with appropriate regulatory sequences required for expression of the nucleic acid sequence in the plant or plant part of choice. Furthermore, the expression construct may comprise a selectable marker useful for identifying host cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences is determined, for example, on the basis of when, where, and how the aspartic acid protease is desired to be expressed. For instance, the expression of the gene encoding an aspartic acid protease of the present invention may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the ³⁵S-CaMV promoter may be used (Franck et al., 1980, Cell 21: 285-294). Organ-specific promoters may be, for example, a promoter from storage sink tissues such as seeds, potato tubers, and fruits (Edwards & Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant and Cell Physiology 39: 885-889), a Vicia faba promoter from the legumin B4 and the unknown seed protein gene from Vicia faba (Conrad et al., 1998, Journal of Plant Physiology 152: 708-711), a promoter from a seed oil body protein (Chen et al., 1998, Plant and Cell Physiology 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al., 1993, Plant Physiology 102: 991-1000, the chlorella virus adenine methyltransferase gene promoter (Mitra and Higgins, 1994, Plant Molecular Biology 26: 85-93), or the aldP gene promoter from rice (Kagaya et al., 1995, Molecular and General Genetics 248: 668-674), or a wound inducible promoter such as the potato pin2 promoter (Xu et al., 1993, Plant Molecular Biology 22: 573-588).

A promoter enhancer element may also be used to achieve higher expression of the enzyme in the plant. For instance, the promoter enhancer element may be an intron, which is placed between the promoter and the nucleotide sequence encoding an aspartic acid protease of the present invention. For instance, Xu et al., 1993, supra disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Presently, Agrobacterium tumefaciens-mediated gene transfer is the method of choice for generating transgenic dicots (for a review, see Hooykas and Schilperoort, 1992, Plant Molecular Biology 19: 15-38). However it can also be used for transforming monocots, although other transformation methods are generally preferred for these plants. Presently, the method of choice for generating transgenic monocots is particle bombardment (microscopic gold or tungsten particles coated with the transforming DNA) of embryonic calli or developing embryos (Christou, 1992, Plant Journal 2: 275-281; Shimamoto, 1994, Current Opinion Biotechnology 5: 158-162; Vasil et al., 1992, Bio/Technology 10: 667-674). An alternative method for transformation of monocots is based on protoplast transformation as described by Omirulleh et al., 1993, Plant Molecular Biology 21: 415-428.

Following transformation, the transformants having incorporated therein the expression construct are selected and regenerated into whole plants according to methods well known in the art.

The present invention also relates to methods for producing an aspartic acid protease of the present invention comprising (a) cultivating a transgenic plant or a plant cell comprising a nucleic acid sequence encoding an aspartic acid protease of the present invention under conditions conducive for production of the aspartic acid protease; and (b) recovering the aspartic acid protease.

Methods of Production

The present invention also relates to methods for producing an aspartic acid protease of the present invention comprising (a) cultivating a strain, which in its wild-type form is capable of producing the aspartic acid protease, to produce a supernatant comprising the aspartic acid protease; and (b) recovering the aspartic acid protese. Preferably, the strain is of the genus Pseudozyma, in particular Pseudozyma sp.

The present invention also relates to methods for producing an aspartic acid protese of the present invention comprising (a) cultivating a host cell under conditions conducive for production of the aspartic acid protease; and (b) recovering the aspartic acid protease.

In the production methods of the present invention, the cells are cultivated in a nutrient medium suitable for production of the aspartic acid protease using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the aspartic acid protese to be expressed and/or isolated. The cultivation takes place in a suitable nutrient medium comprising carbon and nitrogen sources and inorganic salts, using procedures known in the art (see, e.g., references for bacteria and yeast; Bennett, J. W. and LaSure, L., editors, More Gene Manipulations in Fungi, Academic Press, CA, 1991). Suitable media are available from commercial suppliers or may be prepared according to published compositions (e.g., in catalogues of the American Type Culture Collection). If the aspartic acid protease is secreted into the nutrient medium, the aspartic acid proteae can be recovered directly from the medium. If the aspartic acid protease is not secreted, it can be recovered from cell lysates.

The aspartic acid protease may be detected using methods known in the art that are specific for the aspartic acid protease. These detection methods may include use of specific antibodies, formation of an enzyme product, or disappearance of an enzyme substrate. For example, an enzyme assay may be used to determine the activity of the aspartic acid protease. The resulting aspartic acid protease may be recovered by methods known in the art. For example, the aspartic acid protease may be recovered from the nutrient medium by conventional procedures including, but not limited to, centrifugation, filtration, extraction, spray-drying, evaporation, or precipitation.

The aspartic acid proteases of the present invention may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), or extraction (see, e.g., Protein Purification, J. -C. Janson and Lars Ryden, editors, VCH Publishers, New York, 1989).

The present invention is further illustrated by the following non-limiting examples.

Materials and Methods

Molecular cloning techniques are described in J. Sambrook, E. F. Fritsch, and T. Maniatis, 1989, Molecular Cloning, A Laboratory Manual, 2nd Edition, Cold Spring Harbor, N.Y.

The following commercial plasmids/vectors were used: pYES 2.0 (Invitrogen, USA)

The following strains were used for transformantion and protein expression: E. coli. DH10B.

Chemicals used as buffers and substrates were commercial products of at least reagent grade.

The “BSA-BCA assay”

Assay Buffers:

100 mM succinic acid, 100 mM HEPES, 100 mM CHES, 100 mM CABS, 1 mM CaCl₂, 150 mM KCl, 0.01% (v/v) Triton X-100 was adjusted to the pH values 2.0, 3.0, 4.0, 5.0, 6.0, 7.0, 8.0 and 9.0 with HCl or NaOH.

Assay Substrates:

150 mg BSA (Sigma A-7906) was dissolved in 20.0 ml assay buffer and the pH was re-adjusted to the relevant pH (i.e. 2.0, 3.0, 4.0, 5.0, 6.0, 7.0, 8.0 and 9.0). Finally, the assay substrate was filtered through a 0.45 μm filter (Catalogue No. 16555 from Sartorius).

BSA Assay:

400 μl assay substrate (pH 3.0) was placed on ice in an Eppendorf tube. 200 μl aspartic acid protease sample (diluted in assay buffer, pH 3.0) was added.

The assay was initiated by transferring the Eppendorf tube to an Eppendorf thermomixer, which was to set to 37° C. The tube was incubated for 30 minutes in the Eppendorf thermomixer at its highest shaking rate.

The incubation was terminated by transferring the tube back to the ice batch. In the ice batch 150 μl 20% (w/v) TCA (trichloro acetic acid) was added and the tube was vortexed. In order to ensure complete precipitation of protein the sample was then left at room temperature for 15 min after which the mixture was filtered through a 0.45 μm filter (Catalogue No. 16555 from Sartorius). The content of soluble protein (or rather TCA-soluble peptides) in the filtrate was measured relative to a BSA standard using the “BCA assay” (see below) and was used as a measure for the protease activity. A buffer blind (no enzyme) was also included in the assay.

BCA Assay:

The employed assay was PIERCE Cat. No. 23225: BCA protein assay reagent kit. The BCA working solution was made by mixing 50 parts of reagent A with 1 part of reagent B. 200 μl sample (filtrate) was mixed with 2.0 ml BCA working solution. After 30 minutes at 37° C., the sample was cooled to room temperature and OD₄₉₀ was read as a measure for the protein concentration in the sample. Dilutions of BSA were included in the assay as a standard.

OD₄₉₀ values were transformed to concentrations (mg hydrolysis product/ml) by using the BSA standard. The activity can then be determined by dividing the concentration (mg/ml) with the total reaction time (30 min) and multiply the result with the dilution in the BSA assay (3.75=750 μl/200 μl). Thus, one unit in the “BSA-BCA” assay is defined as the amount of protease that gives a 1.0 mg/min response as TCA-soluble peptides in the filtrate.

The specific activity of the protease can be determined as the ratio between the activity of the protease and the protease concentration.

The “BSA-BCA pH-Activity Assay”

This assay was carried out as described above in connection with the “BSA-BCA assay”, the only difference being that the assay was carried out at different pH-values, i.e. at pH values of 2.0, 3.0, 4.0, 5.0, 6.0, 7.0, 8.0 and 9.0.

The “BSA-BCA pH-Stability Assay”

200 μl protease sample (diluted in assay buffers) at pH 2.0, 3.0, 4.0, 5.0, 6.0, 7.0, 8.0 or 9.0 was incubated for 2 hours at 37° C.

Subsequently, 400 μl assay substrate (pH 3.0) was added and, if necessary, pH was adjusted to 3. The protease activity was then measured as described above in connection with the “BSA-BCA assay”.

The residual activity was measured relative to the activity of a protease sample, which was incubated for 2 hours at 5° C. and pH 3.0.

The “BSA-BCA Temperature-Activity Assay”

This assay was carried out as described above in connection with the “BSA-BCA assay”, the only difference being that the assay was carried out at different temperatures, i.e. at temperatures at 15° C., 25° C., 37° C., 50° C., 60° C. and 70° C.

### EXAMPLES

### Example 1a

### Partial Purification of the Pseudozyma aspartic Acid Protease

The Pseudozyma strain was isolated from leaf litter of an unidentified plant from the Guangdong Province, China. The fungi were grown on Potato Dextrose Agar plate (4.5 cm in diameter) in darkness for 6 days at 27° C. and were used for inoculating shake flasks. The plates with fully growing cultures were stored at 4° C. before use.

For enzyme production, 4-6 agar plugs with fully growing fungal cultures on the plates were used to inoculate one shake flask with media A (see below) and was grown for 3-4 days at 27° C. (160 rpm) and subsequently used for inoculating shake flasks with media A, B or C (see below).

About 1 ml cultivated culture broth in media A was used to inoculate each flask with media A, B or C. The inoculated flasks were grown under the following growth conditions:

| | | |
|---|---|---|
| Temperature: | 27° C. | |
| RPM: | Medium B: | stationary |
| | Medium A and B: | 160 rpm |
| Incubation time: | Medium A and B: | 6-7 days |
| | Medium B: | 7-9 days. |

Media
Potato Dextrose Agar:
24 g potato dextrose broth (Difco 0549)
20 g Agar
1000 ml de-ionized water
Autoclaved for 20 mm vat 121° C.
Media A (FG-4);
30 g soymeal, 15 g maltose, 5 g peptone, 1000 ml H₂0, 1 g olive oil (2
drops/flask); 50 ml in 500 ml Erlenmeyer flask with 2 baffles. Autoclaved
for 30 min at 121° C.
Media B per flask:
30 g wheat bran, 45 ml of the following solution:
4 g yeast extract, 1 g KH₂PO₄, 0.5 g MgSO₄7H₂O, 15 g glucose;
1000 ml tap water. Autoclaved for 30 min at 121° C.
Media C (SC media)
40 g soymeal, 20 g cornmeal, 10 g NH₄Cl, 5 g CaCl₂, 4 g
Na₂HPO₄, 1000 ml H₂O, 1 g olive oil (2 drops/flask); pH adjusted to 5.5;
50 ml in 500 ml Erlenmeyer flask with 2 baffles. Autoclaved for 30 min at
121° C.

The culture broths, which were produced in medium A or C were centrifuged for 20 minutes at 10,000 rpm and 4° C. The supernatants were collected and tested for acidic protease activity using an agarose plate assay (see below).

To each flask with media B and fully growing culture, 150 ml tap water was added and homogenized by a sterilized glass rod. The enzyme was extracted by leaving the flask for 4-14 hours at 4° C. The culture broth was then centrifuged for 30 minutes at 8,000 rpm and 4° C. and the supernatant was collected and tested for acidic protease activity by using an agarose plate assay (see below).

In order to get enough sample for enzyme purification and characterization, the strain was grown in 15 shake flasks with media B. 2,200 ml crude enzyme sample was obtained from extraction by adding 150 ml sterilized water into each flask followed by centrifugation. Total protein was precipitated by 100% ammonium sulfate and re-dissolved in phosphate buffer, pH 6.8. 120 ml precipitated sample was obtained after de-salting. 15 ml sample was then placed in a number of 50 ml plastic tubes and lyophilized. The content of one tube was used for further purification and characterization of the aspartic acid protease as described below.

Agarose Plate Assay

The supernatants were tested for protease activity by using the following assay: a) Shake flask containing 2% agarose (Litex LSA) in phosphate-citrate buffer (pH 3) was heated to the boiling point for 5 minutes, where after it was cooled to 55° C.; b) 1% Na-casein was dissolved in phosphate-citrate buffer (pH 3) at 55° C.; c) Equal amount of a) and b) were mixed and poured into 9 cm (diameter) petri dishes and were left for half an hour; d) 4 mm holes (diameter) were punched by a puncher; e) 20 μl sample were applied into the holes in the agarose plates; f) The plates were incubated at 45° C. for 12-16 hours. Enzymatic activity was identified by a clear zone.

### Example 1b

### Final Purification and Characterization of the Pseudozyma aspartic Acid Protease

The lyophilized powder (15 ml) was dissolved in 5 ml 20 mM acetic acid/NaOH, pH 4, and filtered through a 0.45 μm filter. The filtrate was applied to a 300 ml Superdex 75 size exclusion column equilibrated with 50 mM acetic acid/NaOH, 100 mM NaCl, pH 3.5, and the column was eluted with the same buffer. Fractions from the column were analyzed for protease activity by activation of trypsinogen to trypsin at pH 4.0 (see below). The fractions with trypsinogen activation activity were pooled and diluted with de-ionized water to the same conductivity as 20 mM citric acid/NaOH, pH 3.0. The diluted pool was applied to a 14 ml S-Sepharose HP column equilibrated with the same buffer. After washing the column with the equilibration buffer, the protease was eluted with a linear NaCl gradient (0→0.5 M). Protease-containing fractions were pooled and diluted 10 times with de-ionized water and applied to a 5 ml HighTrap S column equilibrated with 20 mM citric acid/NaOH, pH 3.0. After washing the column with the equilibration buffer, the protease was eluted with a linear NaCl gradient (0→0.2 M). Protease-containing fractions were then analyzed by SDS-PAGE and pure fractions were pooled. The resulting product was freezed (−20° C.) in aliquots.

The concentration of the aspartic acid protease of the invention was determined by the BCA assay described above. The concentration of protease was 0.110 mg/ml.

The activity, as determined in the “BSA-BCA assay”, was 0.732 units/ml and, consequently, the specific activity of the aspartic acid protease of the invention was 6.65 units/mg. This value was about 2.5 times higher than the specific activity of the aspartic acid protease II from Aspergillus aculeatus (WO 94/02044) when tested under identical conditions.

In addition, The N-terminal sequence of the enzyme was determined by further purifying the lyophilized powder (see Example 1a) by means of ion exchange chromatography. All fractions were analyzed for aspartic acid protease activity. Four fractions containing the majority of activity were pooled and de-salted. These fractions were subjected to SDS-PAGE, which revealed a clear band with a molecular weight of about 35 kDa. This band was electro-blotted and the N-terminal sequence was determined to be AGTGSVSLTDIQNEELWSGPVK (also shown in SEQ ID No 1, amino acids 1-22).

Trypsinogen Activation:

50 μl protease (diluted in 25 mM acetic acid/NaOH, pH 4) was mixed with 50 μl trypsinogen (1 mg/ml, Sigma T-1143, dissolved in 25 mM acetic acid/NaOH, pH 4—prepared the same day) and incubated for 5 minutes at 25° C. The incubation was stopped and the assay for active trypsin started by adding 100 μl Bz-Arg-pNA substrate (50 mg Sigma B-4875 dissolved in 1.0 ml DMSO and further diluted 100 times in 0.25 M Tris/HCl, pH 8.3) and measuring the increase in OD₄₀₅ as the protease activity. If the increase in OD₄₀₅ was larger than 0.2 OD_{405/min}, the protease was diluted further.

### Example 2

### pH Activity Profile

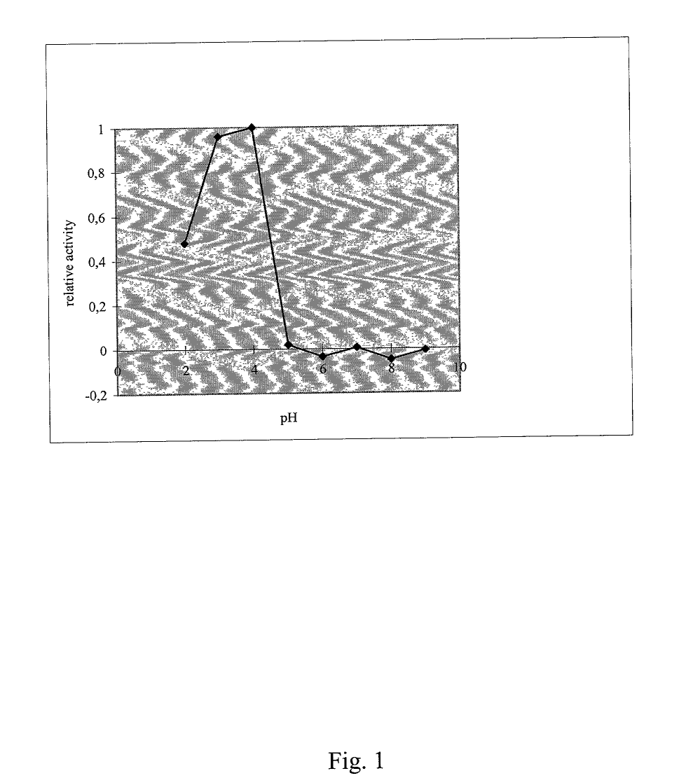

The experiments were carried out as described in the “BSA-BCA pH-activity assay” described above. As it appears from the graph shown in FIG. 1, the pH optimum of the aspartic acid protease of the invention is in the range from about 3 to about 4. Furthermore, it can be seen that the aspartic acid protease of the invention possesses essentially same activity at both pH 3 and 4, whereas the activity declines rapidly at pH values above 4-4.5

### Example 3

### pH stability profile

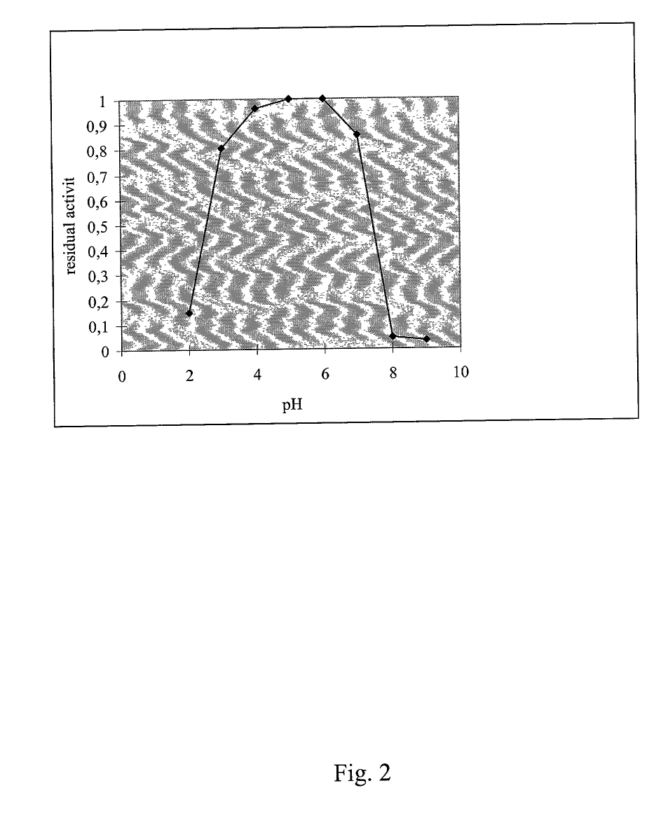

The experiments were carried out as described in the “BSA-BCA pH-stability assay” described above. As it appears from the graph shown in FIG. 2, the aspartic acid protease according to the invention remains stable after incubation at pH 3-7 for 2 hours at 37° C.

### Example 4

### Temperature Profile

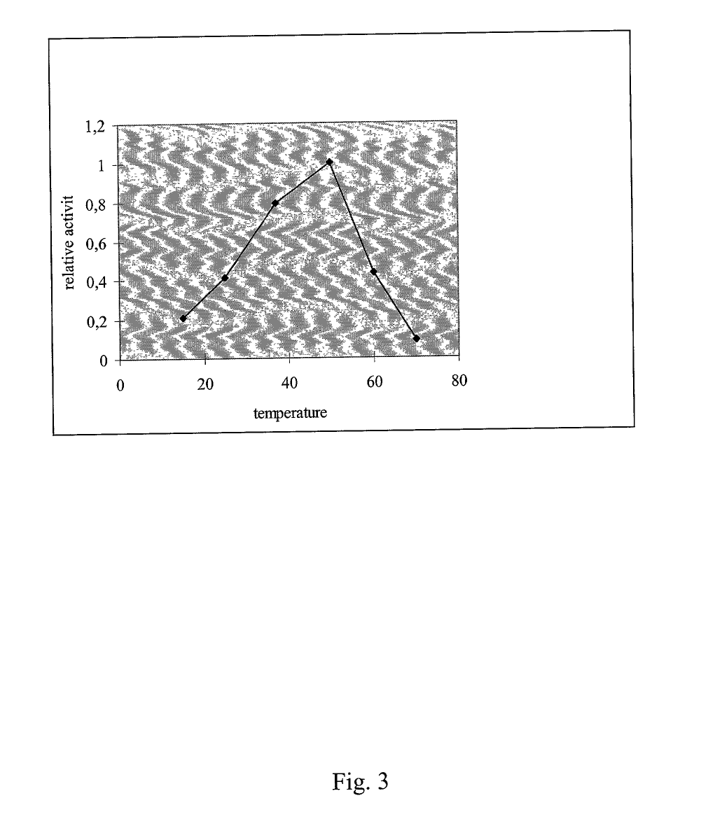

The experiments were carried out as described in the “BSA-BCA temperature-activity assay” described above. As it appears from the graph shown in FIG. 3, the aspartic acid protease according to the invention has the highest activity in the temperature range of from 35 to 55° C. The highest activity appears at a temperature of about 50° C.

### Example 5

### Example 5a

### Fungal Strains and Growth Conditions

The Pseudozyma sp. strain was cultivated in shake flasks as described in Example 1 a, and subsequently the fungal mycelium was harvested. The harvested mycelia were immediately frozen in liquid N₂ and stored at −80° C.

### Example 5b

### Construction of a EcoRI/NotI-Directional cDNA Library from Pseudozyma sp.

Total RNA was prepared by extraction with guanidinium thiocyanate followed by ultracentrifugation through a 5.7 M CsCl cushion (Chirgwin et al., 1979, Biochemistry 18: 5294-5299) using the following modifications. The frozen mycelium was ground in liquid N₂ to a fine powder with a mortar and a pestle, followed by grinding in a precooled coffee mill, and immediately suspended in 5 volumes of RNA extraction buffer (4 M guanidinium thiocyanate, 0.5% sodium laurylsarcosine, 25 mM sodium citrate pH 7.0, 0.1 M β-mercaptoethanol). The mixture was stirred for 30 minutes at room temperature and centrifuged (20 minutes at 10 000 rpm, Beckman) to pellet the cell debris. The supernatant was collected, carefully layered onto a 5.7 M CsCl cushion (5.7 M CsCl, 10 mM EDTA, pH 7.5, 0.1% DEPC; autoclaved prior to use) using 26.5 ml supernatant per 12.0 ml of CsCl cushion, and centrifuged to obtain the total RNA (Beckman, SW 28 rotor, 25 000 rpm, room temperature, 24 hours). After centrifugation the supernatant was carefully removed and the bottom of the tube containing the RNA pellet was cut off and rinsed with 70% ethanol. The total RNA pellet was transferred to an Eppendorf tube, suspended in 500 μl of TE, pH 7.6 (if difficult, heat occasionally for 5 minutes at 65° C.), phenol extracted, and precipitated with ethanol for 12 hours at −20° C. (2.5 volumes of ethanol, 0.1 volume of 3M sodium acetate pH 5.2). The RNA was collected by centrifugation, washed in 70% ethanol, and resuspended in a minimum volume of DEPC. The RNA concentration was determined by measuring OD_{260/280}.

The poly(A)⁺ RNA was isolated by oligo(dT)-cellulose affinity chromatography (Aviv & Leder, 1972, Proceedings of the National Academy of Sciences USA 69: 1408-1412). A total of 0.2 g of oligo(dT) cellulose (Boehringer Mannheim, Indianapolis, Ind.) was preswollen in 10 ml of lx of column loading buffer (20 mM Tris-Cl, pH 7.6, 0.5 M NaCl, 1 mM EDTA, 0.1% SDS), loaded onto a DEPC-treated, plugged plastic column (Poly Prep Chromatography Column, BioRad, Hercules, Calif.), and equilibrated with 20 ml of 1× loading buffer. The total RNA (1-2 mg) was heated at 65° C. for 8 minutes, quenched on ice for 5 minutes, and after addition of 1 volume of 2× column loading buffer to the RNA sample loaded onto the column. The eluate was collected and reloaded 2-3 times by heating the sample as above and quenching on ice prior to each loading. The oligo(dT) column was washed with 10 volumes of 1× loading buffer, then with 3 volumes of medium salt buffer (20 mM Tris-Cl, pH 7.6, 0.1 M NaCl, 1 mM EDTA, 0.1% SDS), followed by elution of the poly(A)⁺ RNA with 3 volumes of elution buffer (10 mM Tris-Cl, pH 7.6, 1 mM EDTA, 0.05% SDS) preheated to 65° C., by collecting 500 μl fractions. The OD₂₆₀ was read for each collected fraction, and the mRNA containing fractions were pooled and ethanol precipitated at −20° C. for 12 hours. The poly(A)⁺ RNA was collected by centrifugation, resuspended in DEPC-DIW and stored in 5-10 μg aliquots at −80° C.

Double-stranded cDNA was synthesized from 5 μg of Pseudozyma sp. poly(A)⁺ RNA by the RNase H method (Gubler and Hoffinan 1983, supra; Sambrook et al., 1989, supra) using a hair-pin modification. The poly(A)⁺ RNA (5 μg in 5 μl of DEPC-treated water) was heated at 70° C. for 8 minutes in a pre-siliconized, RNase-free Eppendorf tube, quenched on ice, and combined in a final volume of 50 μl with reverse transcriptase buffer (50 mM Tris-Cl pH 8.3, 75 mM KCl, 3 mM MgCl₂, 10 mM DTT) containing 1 mM of dATP, dGTP and dTTP, and 0.5 mM of 5-methyl-dCTP, 40 units of human placental ribonuclease inhibitor, 4.81 μg of oligo(dT)18-NotI primer (Amersham-Pharmacia Biotech, Uppsala, Sweden) and 1000 units of SuperScript II reverse transcriptase (Gibco-BRL, USA).

First-strand cDNA was synthesized by incubating the reaction mixture at 45° C. for 1 hour. After synthesis, the mRNA:cDNA hybrid mixture was gel filtrated through a Pharmacia MicroSpin S-400 HR spin column according to the manufacturer's instructions.

After the gel filtration, the hybrids were diluted in 250 μl of second strand buffer (20 mM Tris-Cl pH 7.4, 90 mM KCl, 4.6 mM MgCl₂, 10 mM (NH₄)₂SO₄, 0.16 mM βNAD⁺) containing 200 μM of each dNTP, 60 units of E. coli DNA polymerase I (Pharmacia, Uppsala, Sweden), 5.25 units of RNase H, and 15 units of E. coli DNA ligase. Second strand cDNA synthesis was performed by incubating the reaction tube at 16° C. for 2 hours, and an additional 15 minutes at 25° C. The reaction was stopped by addition of EDTA to 20 mM final concentration followed by phenol and chloroform extractions.

The double-stranded cDNA was ethanol precipitated at −20° C. for 12 hours by addition of 2 volumes of 96% ethanol and 0.2 volume of 10 M ammonium acetate, recovered by centrifugation, washed in 70% ethanol, dried (SpeedVac), and resuspended in 30 μl of Mung bean nuclease buffer (30 mM sodium acetate pH 4.6, 300 mM NaCl, 1 mM ZnSO₄, 0.35 mM dithiothreitol, 2% glycerol) containing 25 units of Mung bean nuclease. The single-stranded hair-pin DNA was clipped by incubating the reaction at 30° C. for 30 minutes, followed by addition of 70 μl of 10 mM Tris-Cl, pH 7.5, 1 mM EDTA, phenol extraction, and ethanol precipitation with 2 volumes of 96% ethanol and 0.1 volume 3 M sodium acetate pH 5.2 on ice for 30 minutes.

The double-stranded cDNAs were recovered by centrifugation (20,000 rpm, 30 minutes), and blunt-ended with T4 DNA polymerase in 30 μl of T4 DNA polymerase buffer (20 mM Tris-acetate, pH 7.9, 10 mM magnesium acetate, 50 mM potassium acetate, 1 mM dithiothreitol) containing 0.5 mM of each dNTP, and 5 units of T4 DNA polymerase by incubating the reaction mixture at +16° C. for 1 hour. The reaction was stopped by addition of EDTA to 20 mM final concentration, followed by phenol and chloroform extractions and ethanol precipitation for 12 h at −20° C. by adding 2 volumes of 96% ethanol and 0.1 volume of 3M sodium acetate pH 5.2.

After the fill-in reaction the cDNAs were recovered by centrifugation as above, washed in 70% ethanol, and the DNA pellet was dried in a SpeedVac. The cDNA pellet was resuspended in 25 μl of ligation buffer (30 mM Tris-Cl, pH 7.8, 10 mM MgCl₂, 10 mM dithiothreitol, 0.5 mM ATP) containing 2 μg EcoRI adaptors (0.2 μg/μl, Pharmacia, Uppsala, Sweden) and 20 units of T4 ligase by incubating the reaction mix at 16° C. for 12 hours. The reaction was stopped by heating at 65° C. for 20 minutes, and then placed on ice for 5 minutes. The adapted cDNA was digested with NotI by addition of 20 μl autoclaved water, 5 μl of 10× NotI restriction enzyme buffer and 50 units of NotI, followed by incubation for 3 hours at 37° C. The reaction was stopped by heating the sample at 65° C. for 15 minutes. The cDNAs were size-fractionated by agarose gel electrophoresis on a 0.8% SeaPlaque GTG low melting temperature agarose gel (FMC, Rockland, Me.) in 1× TBE (in autoclaved water) to separate unligated adaptors and small cDNAs. The gel was run for 12 hours at 15 V, and the cDNA was size-selected with a cut-off at 0.7 kb by cutting out the lower part of the agarose gel. Then a 1.5% agarose gel was poured in front of the cDNA-containing gel, and the double-stranded cDNAs were concentrated by running the gel backwards until it appeared as a compressed band on the gel. The cDNA-containing gel piece was cut out from the gel and the cDNA was extracted from the gel using the GFX gel band purification kit (Amersham, Arlington Heights, Ill.) as follows. The trimmed gel slice was weighed in a 2 ml Biopure Eppendorf tube, then 10 ml of Capture Buffer was added for each 10 mg of gel slice, the gel slice was dissolved by incubation at 60° C. for 10 minutes, until the agarose was completely solubilized, the sample at the bottom of the tube by brief centrifugation. The melted sample was transferred to the GFX spin column placed in a collection tube, incubated at 25° C. for 1 minute, and then spun at full speed in a microcentrifuge for 30 seconds. The flow-through was discarded, and the column was washed with 500 μl of wash buffer, followed by centrifugation at full speed for 30 seconds. The collection tube was discarded, and the column was placed in a 1.5 ml Eppendorf tube, followed by elution of the cDNA by addition of 50 μl of TE pH 7.5 to the center of the column, incubation at 25° C. for 1 minute, and finally by centrifugation for 1 minute at maximum speed. The eluted cDNA was stored at −20° C. until library construction.

A plasmid DNA preparation for a EcoRI-NotI insert-containing pYES2.0 cDNA clone, was purified using a QIAGEN Tip-100 according to the manufacturer's instructions (QIAGEN, Valencia, Calif.). A total of 10 μg of purified plasmid DNA was digested to completion with NotI and EcoRI in a total volume of 60 μl by addition of 6 μl of 10× NEBuffer for EcoRI (New England Biolabs, Beverly, Mass.), 40 units of NotI, and 20 units of EcoRI followed by incubation for 6 hours at 37° C. The reaction was stopped by heating the sample at 65° C. for 20 minutes. The digested plasmid DNA was extracted once with phenol-chloroform, then with chloroform, followed by ethanol precipitation for 12 hours at −20° C. by adding 2 volumes of 96% ethanol and 0.1 volume of 3 M sodium acetate pH 5.2. The precipitated DNA was resuspended in 25 μl of 1× TE pH 7.5, loaded on a 0.8% SeaKem agarose gel in 1× TBE, and run on the gel for 3 hours at 60 V. The digested vector was cut out from the gel, and the DNA was extracted from the gel using the GFX gel band purification kit (Amersham-Pharmacia Biotech, Uppsala, Sweden) according to the manufacturer's instructions. After measuring the DNA concentration by OD_{260/280}, the eluted vector was stored at −20° C. until library construction.

To establish the optimal ligation conditions for the cDNA library, four test ligations were carried out in 10 μl of ligation buffer (30 mM Tris-Cl pH 7.8, 10 mM MgCl₂, 10 MM DTT, 0.5 mM ATP) containing 7 μl of double-stranded cDNA, (corresponding to approximately 1/10 of the total volume in the cDNA sample), 2 units of T4 ligase, and 25 ng, 50 ng and 75 ng of EcoRI-NotI cleaved pYES2.0 vector, respectively (Invitrogen). The vector background control ligation reaction contained 75 ng of EcoRI-NotI cleaved pYES2.0 vector without cDNA. The ligation reactions were performed by incubation at 16° C. for 12 hours, heated at 65° C. for 20 minutes, and then 10 μl of autoclaved water was added to each tube. One μl of the ligation mixtures was electroporated (200 W, 2.5 kV, 25 mF) to 40 μl electrocompetent E. coli DH10B cells (Life Technologies, Gaithersburg, Md.). After addition of 1 ml SOC to each transformation mix, the cells were grown at 37° C. for 1 hour, 50 μl and 5 μl from each electroporation were plated on LB plates supplemented with ampicillin at 100 μg per ml and grown at 37° C. for 12 hours. Using the optimal conditions, a Pseudozyma sp. cDNA library containing 5×10⁶ independent colony forming units was established in E. coli, with a vector background of ca. 1%. The cDNA library was stored as (1) individual pools (25,000 c.f.u./pool) in 20% glycerol at −80° C.; (2) cell pellets of the same pools at −20° C.; (3) Qiagen purified plasmid DNA from individual pools at −20° C. (Qiagen Tip 100); and (4) directional, double-stranded cDNA at −20° C.

### Example 5c

### Generation of a cDNA Probe for the Acidic Aspartic Acid Protease using PCR

Ca. twenty (20) nanograms of directional, double-stranded cDNA from Pseudozyma sp. was PCR amplified using 200 pmol of a degenerate deoxyinosine-containing oligonucleotide primer, corresponding to a peptide within the NH2-terminus of the purified aspartic protease (5′-ACI GA(C/T) ATI CA(A/G) AA(C/T) GA(A/G) GA(A/G) (C/T)TI TGG-3′) (SEQ ID NO:3) combined with 200 pmol of the cDNA anchor primer (5′-GGC CGC AGG AAT TTT TTT T-3′) (SEQ ID NO:4), a PTC-200 Peltier Thermal cycler (MJ Research, USA) and 2.5 units of Taq polymerase (Perkin-Elmer, USA). Thirty cycles of PCR were performed using a cycle profile of denaturation at 94° C. for 30 sec, annealing at 50° C. for 1 min, and extension at 72° C. for 2 min. The amplification products were analyzed by electrophoresis in a 1% agarose gel, and subsequently a ca. 0.9 kb PCR fragment was extracted from the gel using the GFX gel band purification kit (Amersham-Pharmacia Biotech, Uppsala, Sweden) according to the manufacturer's instructions. The purified PCR fragment was sequenced directly by the dideoxy chain-termination method (Sanger, F., Nicklen, S., and Coulson, A. R. (1977) Proc. Natl. Acad. Sci. U.S. A. 74, 5463-5467) using 300 ng of GFX-purified template, the Taq deoxy-terminal cycle sequencing kit (Perkin-Elmer, USA), fluorescent labeled terminators and 5 pmol of the degenerate deoxyinosine-containing oligonucleotide primer (5′-ACI GA(C/T) ATI CA(A/G) AA(C/T) GA(A/G) GA(A/G) (C/T)TI TGG-3′) (SEQ ID NO:3). Analysis of the sequence data was performed according to Devereux et al., 1984 (Devereux, J., Haeberli, P., and Smithies, 0. (1984) Nucleic Acids Res. 12, 387-395).

### Example 5d

### Screening of the cDNA Library

20 000 colony-forming units from the Pseudozyma sp. cDNA library constructed in pYES 2.0 were screened by colony hybridization (Sambrook et al. (1989) Molecular cloning: A laboratory manual, Cold Spring Harbor lab., Cold Spring Harbor, N.Y.) in E. coli using a random-primed (Feinberg, A. P., and Vogelstein, B. (1983) Anal. Biochem. 132, 6-13) ³²P-labeled (>1×10⁹ cpm/μg) PCR product for the acidic aspartic protease as a probe. The hybridizations were carried out in 2× SSC (Sambrook et al. (1989) Molecular cloning: A laboratory manual, Cold Spring Harbor lab., Cold Spring Harbor, N.Y.), 5× Denhardt's solution (Sambrook et al. (1989) Molecular cloning: A laboratory manual, Cold Spring Harbor lab., Cold Spring Harbor, N.Y.), 0.5% (w/v) SDS, 100 μg/ml denatured salmon sperm DNA for 20 h at 65° C. followed by washes in 5× SSC at 25° C. (2×15 min), 2× SSC, 0.5% SDS at 65° C. (30 min), 0.2× SSC, 0.5% SDS at 65° C. (30 min) and finally in 5× SSC (2×15 min) at 25° C. Screening of the Pseudozyma sp. cDNA library yielded 12 strongly hybridizing clones, which were further analyzed by sequencing the cDNA ends with pYES forward and reverse polylinker primers (Invitrogen, USA), and determining the nucleotide sequence of the longest aspartic protease cDNA (designated pC1PRT1193) from both strands.

### Example 5e

### Nucleotide Sequence Analysis

The nucleotide sequence of the full-length Pseudozyma sp. aspartic protease cDNA clone pC1PRT1193 was determined from both strands by the dideoxy chain-termination method (Sanger, F., Nicklen, S., and Coulson, A. R. (1977) Proc. Natl. Acad. Sci. U.S.A. 74, 5463-5467) using 500 ng of Qiagen-purified template (Qiagen, USA), the Taq deoxy-terminal cycle sequencing kit (Perkin-Elmer, USA), fluorescent labeled terminators and 5 pmol of either pYES 2.0 polylinker primers (Invitrogen, USA) or synthetic oligonucleotide primers. Analysis of the sequence data was performed according to Devereux et al., 1984 (Devereux, J., Haeberli, P., and Smithies, O. (1984) Nucleic Acids Res. 12, 387-395). The obtained sequence is shown as SEQ ID NO:1 herein.

The 1342-bp PRT1193 cDNA contains a 1185-bp open reading frame initiating at nucleotide position 65, and terminating with a TGA stop codon at nucleotide position 1247, thus predicting a 394-residue precursor polypeptide of 41 090 Da. The open reading frame is preceeded by a 64-bp 5′ non-coding region, and followed by a 43-bp 3′ non-coding region and a poly(A) tail.

### Example 6

### Heterologous expression in Aspergillius oryzae

Transformation of Aspergillus oryzae

Transformation of Aspergillus oryzae was carried out as described by Christensen et al., (1988), Biotechnology 6, 1419-1422.

Construction of the Aspartic Acid Protease Expression Cassette for Aspergillus Expression

Plasmid DNA was isolated from the Pseudozyma sp. aspartic protease cDNA clone pC1PRT1193 using standard procedures and analyzed by restriction enzyme analysis. The cDNA insert was excised using appropriate restriction enzymes and ligated into the Aspergillus expression vector pHD414, which is a derivative of the plasmid p775 (described in EP 0 238 023). The construction of pHD414 is further described in WO 93/11249.

Transformation of Aspergillus oryzae or Aspergillus niger

General Procedure:

100 ml of YPD (Sherman et al., Methods in Yeast Genetics, Cold Spring Harbor Laboratory, 1981) is inoculated with spores of A. oryzae or A. niger and incubated with shaking at 37° C. for about 2 days. The mycelium is harvested by filtration through miracloth and washed with 200 ml of 0.6 M MgSO₄. The mycelium is suspended in 15 ml of 1.2 M MgSO₄. 10 mM NaH₂PO₄, pH=5.8. The suspension is cooled on ice and 1 ml of buffer containing 120 mg of Novozym 234 is added. After 5 minutes 1 ml of 12 mg/ml BSA is added and incubation with gentle agitation continued for 1.5-2.5 hours at 37° C. until a large number of protoplasts is visible in a sample inspected under the microscope. The suspension is filtered through miracloth, the filtrate transferred to a sterile tube and overlayered with 5 ml of 0.6 M sorbitol, 100 mM Tris-HCl, pH=7.0. Centrifugation is performed for 15 minutes at 100 g and the protoplasts are collected from the top of the MgSO₄ cushion. 2 volumes of STC are added to the protoplast suspension and the mixture is centrifugated for 5 minutes at 1000 g. The protoplast pellet is resuspended in 3 ml of STC and repelleted. This is repeated. Finally the protoplasts are resuspended in 0.2-1 ml of STC. 100 μl of protoplast suspension is mixed with 5-25 μg of the appropriate DNA in 10 μl of STC. Protoplasts are mixed with p3SR2 (an A. nidulans amdS gene carrying plasmid). The mixture is left at room temperature for 25 minutes. 0.2 ml of 60% PEG 4000. 10 mM CaCl₂ and 10 mM Tris-HCl, pH 7.5 is added and carefully mixed (twice) and finally 0.85 ml of the same solution is added and carefully mixed. The mixture is left at room temperature for 25 minutes, spun at 2500 g for 15 minutes and the pellet is resuspended in 2 ml of 1.2 M sorbitol. After one more sedimentation the protoplasts are spread on the appropriate plates. Protoplasts are spread on minimal plates to inhibit background growth. After incubation for 4-7 days at 37° C. spores are picked and spread for single colonies. This procedure is repeated and spores of a single colony after the second re-isolation is stored as a defined transformant.

Purification of the Aspergillus oryzae Transformants

Aspergillus oryzae colonies are purified through conidial spores on AmdS+-plates (+0,01% Triton X-100).

Deposit of Biological Material

The following biological material has been deposited under the terms of the Budapest Treaty with the Deutsche Sammlung von Mikroorganismen und Zellkulturen GmbH, Mascheroder Weg 1 B, D-38124 Braunschweig, Germany, and given the following accession number:

| Deposit | Accession Number | Date of Deposit |
|---|---|---|
| E. coli | DSM 13470 | 2 May 2000 |

The deposit was made by Novo Nordisk A/S and was later assigned to Novozymes A/S.

1. An aspartic acid protease, selected from the group consisting of:
(a) an aspartic acid protease having an amino acid sequence which has at least 40% identity with the amino acid sequence shown as amino acids 1 to 300 of SEQ ID NO:2;
(b) an aspartic acid protease which is encoded by a nucleic acid sequence which hybridizes under low stringency conditions with
(i) a complementary strand of the nucleic acid sequence shown as nucleotides 347 to 1246 of SEQ ID NO:1, or
(ii) a subsequence of (i) of at least 100 nucleotides;
(c) an aspartic acid protease encoded by the aspartic acid protease encoding part of the DNA sequence cloned into a plasmid present in Escherichia coli DSM 13470, or a variant thereof having at least 40% identity to said aspartic acid protease;
(d) an aspartic acid protease having a relative activity of at least 0.75 throughout the pH range from 3 to 4, when tested at 37° C. for 30 min in the “BSA-BCA pH-activity assay” described herein;
(e) an aspartic acid protease having a residual activity of at least 0.70 after incubation for 2 hours at 37° C. throughout the pH range from 3 to 7 and subsequently tested at 37° C. for 30 min at pH 3 in the “BSA-BCA pH-stability assay” described herein; and
(f) an aspartic acid protease having a specific activity (units/mg protease) of at least 4.0 when tested for 30 min at pH 3 and 37° C. in the “BSA-BCA assay” described herein.

2. A protease according to claim 1, having an amino acid sequence which has at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identity with the amino acid sequence shown as amino acids 1 to 300 of SEQ ID NO:2.

3. A protease according to claim 1, comprising the amino acid sequence shown as amino acids 1 to 300 of SEQ ID NO:2.

4. A protease according to claim 3, consisting of the amino acid sequence shown as amino acids 1 to 300 of SEQ ID NO:2.

5. A protease according to claim 1, wherein the protease has at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identity with the aspartic acid protease encoded by the aspartic acid protease encoding part of the DNA sequence cloned into a plasmid present in Escherichia coli DSM 13470.

6. A protease according to claim 1, which is encoded by a nucleic acid sequence which hybridizes under medium stringency conditions, preferably under high stringency conditions, with
(i) a complementary strand of the nucleic acid sequence shown as nucleotides 347 to 1246 of SEQ ID NO: 1, or
(ii) a subsequence of (i) of at least 100 nucleotides;

7. A protease according to claim 1, wherein the protease is a variant comprising one or more substitutions, deletions and/or insertions compared to the protease having the amino acid sequence shown as amino acids 1 to 300 of SEQ ID NO:2

8. A protease according to claim 1(d), wherein the protease has a relative activity of at least 0.80, preferably at least 0.85, in particular at least 0.90.

9. A protease according to claim 1(e), wherein the protease has a residual activity of at least 0.75.

10. A protease according to claim 1(f), wherein the protease has a specific activity (units/mg protease) of at least 4.5, such as at least 5.0, preferably at least 5.5, more preferably at least 6.0, in particular at least 6.5, when tested for 30 min at pH 3 and 37° C. in the “BSA-BCA assay” described herein.

11. A protease according to any of the preceding claims, wherein the protease has a relative activity of at least 0.50, preferably at least 0.60, throughout the temperature range from 35 to 55° C., such as throughout the temperature range from 40 to 55° C., when tested at pH 3 for 30 min in the “BSA-BCA temperature-activity assay” described herein.

12. An isolated nucleic acid sequence comprising a nucleic acid sequence which encodes for the aspartic acid protease defined in any of claims 1-11.

13. An isolated nucleic acid sequence encoding an aspartic acid protease, selected from the group consisting of:
(a) a nucleic acid sequence having at least 40% identity with the nucleic acid sequence shown as nucleotides 347 to 1246 of SEQ ID NO: 1;
(b) a nucleic acid sequence which hybridizes under low stringency conditions with
(i) a complementary strand of the nucleic acid sequence shown as nucleotides 347 to 1246 of SEQ ID NO:1, or
(ii) a subsequence of (i) of at least 100 nucleotides; and
(c) the aspartic acid encoding part of the DNA sequence which has been cloned into a plasmid present in Escherichia coli DSM 13470, or a variant thereof having at least 40% identity to said DNA sequence;
or an isolated nucleic acid sequence which is the complementary strand of (a), (b) or (c).

14. The nucleic acid sequence of claim 13, having a nucleic acid sequence which has at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identity with the nucleic acid sequence shown as nucleotides 347 to 1246 of SEQ ID NO:1.

15. The nucleic acid sequence of claim 13, having a nucleic acid sequence which has at least 45%, at least 50%, at least 55%, at least 60%, at least 65%, at least 70%, at least 75%, at least 80%, at least 85%, at least 90%, at least 95%, at least 96%, at least 97%, at least 98%, or at least 99% identity with the the aspartic acid protease encoding part of the DNA sequence which has been cloned into a plasmid present in Escherichia coli DSM 13470.

16. A nucleic acid construct comprising the nucleic acid sequence of any of claims 12-15 operably linked to one or more control sequences capable of directing the expression of the aspartic acid protease in a suitable expression host.

17. A recombinant expression vector comprising the nucleic acid construct of claim 16, a promoter, and transcriptional and translational stop signals.

18. A recombinant host cell comprising the nucleic acid construct of claim 16.

19. A method for producing an aspartic acid protease as defined in any of claims 1-11, the method comprising:
(a) cultivating a strain from the genus Pseudozyma, preferably from the species Pseudozyma sp. to produce a supernatant comprising the aspartic acid protease; and
(b) recovering the aspartic acid protease.

20. A method for producing an aspartic acid protease as defined in any of claims 1-11, the method comprising:
(a) cultivating a recombinant host cell as defined in claim 18 under conditions conducive to the production of the aspartic acid protease; and
(b) recovering the aspartic acid protease.

21. Use of an aspartic acid protease as defined in any of claims 1-11 during yeast fermentation.

22. Use according to claim 21, wherein the yeast fermentation is for alcohol production.
